(19) United States
(12) Patent Application Publication
(10) Pub. No.: US 2010/0311912 A1
(43) Pub. Date: Dec. 9, 2010

(54) ANTIMICROBIAL SURFACES

(76) Inventors: Hans J Griesser, Gumeracha (AU); Chi P. NDI, Adelaide (AU); Susan J. Semple, Mylor (AU); Hardi Ys, Adelaide (AU)

Correspondence Address: Olson & Cepuritis, LTD., 20 NORTH WACKER DRIVE, 36TH FLOOR, CHICAGO, IL, 60606, US

(21) Appl. No.: 12/865,045

(22) PCT Filed: Jan. 29, 2009
(86) PCT No.: PCT/AU2009/000094

§ 371 Date: Jul. 28, 2010

Related U.S. Application Data
(60) Provisional application No. 61/024,325, filed on Jan. 29, 2008.

(51) Int. Cl. C08F 214/18; C07C 49/303; B05D 1/36; C08F 2/46

(52) U.S. Cl. 525/326.2; 568/326; 427/402; 427/488

(57) ABSTRACT

The present invention relates to substrates comprising a surface to which a compound is immobilized, methods for immobilizing the compounds to substrates, as well as devices, such as medical devices, comprising said substrates. Also described are compounds suitable for use in preparing such substrates. Compounds immobilized on the substrates and compounds for preparing such substrates include antimicrobial and/or anti-inflammatory compounds according to Formulas (I), (II), (III), (IV), (V), (VI), and (VII) described herein.

### INCORPORATION BY REFERENCE

This patent application claims priority from:
U.S. 61/024,325 titled “ANTIMICROBIAL SURFACES” filed on 29 Jan. 2008.

The entire content of this application is hereby incorporated by reference.

### FIELD OF THE INVENTION

The present invention relates to substrates, particularly solid substrates, comprising a surface to which an antimicrobial compound is immobilised. Such substrates are suitable for use in, for example, medical devices.

### BACKGROUND OF THE INVENTION

The arid-land plant genus Eremophila (Myoporaceae), native only to Australia, includes over 200 different species (Chinnock R J, 2007). A small proportion of Eremophila species have recorded medicinal uses in traditional Aboriginal cultures for symptoms possibly indicative of bacterial infection such as skin sores and sore throat (Barr A et al., 1993; Ghisalberti E L, 1994).

Previously, preparations from a small number of recorded medicinal Eremophila species including E. duttonii and E. alternifolia have been shown to have antibacterial activity against Gram-positive bacteria (Palombo, E A and Semple, S J, 2001; Shah A, et al., 2004). Recently, in a study focussing on Eremophila species producing large quantities of leaf resin, the present applicant showed that extracts of a number of other Eremophila species unreported in traditional medicine, including E. serrulata, have antimicrobial activity against Gram-positive bacteria including clinical isolates of multidrug-resistant Staphylococcus aureus (MRSA) (Ndi et al., 2007). The resin from a number of Eremophila species, extractable with organic solvents such as diethyl ether and acetone (Ghisalberti, E L, 1994) has been the subject of previous chemical structural investigation with a large variety of novel diterpenoids and other chemical compounds of unusual structures isolated (Ghisalberti, E L, 1995). However, very little is known about the compounds responsible for the antimicrobial activity of these plants.

The isolation, structural elucidation, and antimicrobial activity of several diterpenoid antimicrobial compounds derived form Eremophila species is described herein. Further, methods for immobilising described compounds onto a surface of, for example, a solid material are described. These methods were surprisingly found to retain the antimicrobial activity of the compounds.

Infection is a common cause of prolonged patient recovery time following minor or major surgeries. Indeed, approximately 8% of joint replacement surgeries result in infection, and cause extended hospitalisation of patients. Further, many more reported and unreported infections result from the short term implantation of medical devices within the body. Catheter use is a common cause of infection in the hospital setting, with Staphylococcus epidermis the most common cause of catheter-related infection.

Preventative measures may be implemented to avoid the onset of infection and have proved effective in reducing the incidence of infection following surgeries involving the implantation of medical devices. Traditionally these have included the systemic administration of antibiotics to the patient before and/or after surgery. However, in surgeries involving the implantation of medical devices (eg replacement joints), it has been shown that microorganisms are capable of colonising the surface of the implant and produce an extracellular slimy biofilm, which offers the microorganism protection from conventional antimicrobial agents, such as systemically administered antibiotics (Darrouiche, R O, 2004). Consequently, there has been much work recently undertaken on the prevention of microbial colonisation of the surface of medical devices. The immobilisation of certain antibiotic compounds to the surface of implantable medical devices has been shown to be one possible strategy for combating the current incidence of post implant surgery infection.

### SUMMARY OF THE INVENTION

In a first aspect, the present invention provides a substrate comprising at least one surface wherein said surface comprises a compound according to Formula (I) immobilised thereto:

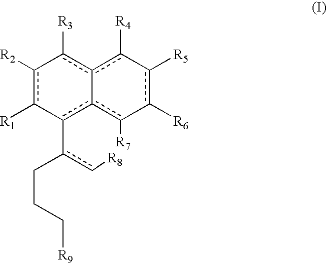

wherein
R₁, R₂, R₃, R₄, R₅, R₆ and R₉ are each independently selected from the group consisting of hydrogen, halogen, hydroxyl, formyl, alkyl, alkoxy, oxoalkyl, alkenyl, aryl, arylalkylarylalkyl, carbonyl, carboxyl, aldehyde, carboxylate, cyano, ester, ether, carboxamide, amide, amine, sulfo, and sulfhydryl residues, their acids or salts, or derivatives thereof; and
R₇ and R₈ are each independently selected from the group consisting of hydrogen, halogen, hydroxyl, formyl, alkyl, alkoxy, oxoalkyl, alkenyl, aryl, alkyaryl, carbonyl, carboxyl, aldehyde, carboxylate, cyano, ester, ether, carboxamide, amide, amine, sulfo, and sulfhydryl residues, their acids or salts, or derivatives thereof, or together form a C₁₋₃ or heteroatom bridge to thereby form a cyclic or heterocyclic group.

Preferably, at least one of R₁, R₂, R₃, R₄, R₅, R₆ and R₉ represents a linking residue to the surface of the substrate, with linking through R₆ or R₉ especially preferred. Preferably, the compound is covalently linked to the substrate surface. In some embodiments, the linking residue(s) may represent a derivative of a functional group (such as an amine group) provided on the surface of the substrate for the purpose of linking a compound according to Formula (I). Such functional groups may, in turn, have been provided on the substrate surface via a binding intermediary (eg an intermediate layer).

Preferably, the compound is according to Formula (II) or (III):

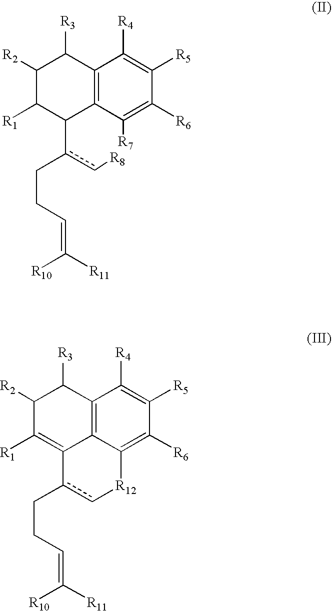

wherein
R₁, R₂, R₃, R₄, R₅, R₆, R₇, R₈, R₁₀ and R₁₁ are each independently selected from the group consisting of hydrogen, halogen, hydroxyl, formyl, alkyl, alkoxy, oxoalkyl, alkenyl, aryl, arylalkylarylalkyl, carbonyl, carboxyl, aldehyde, carboxylate, cyano, ester, ether, carboxamide, amide, amine, sulfo, and sulfhydryl residues, their acids or salts, or derivatives thereof; and
R₁₂ is selected from the group consisting of C₁₋₃ alkyl, C₁₋₃ alkoxy, C₁₋₃ oxoalkyl, C₁₋₃ alkenyl, C₁₋₃ carbonyl, C₁₋₃ carboxyl, C₁₋₃ aldehyde, C₁₋₃ carboxylate, C₁₋₃ cyano, C₁₋₃ ester, C₁₋₃ ether, C₁₋₃ carboxamide, C₁₋₃ amide, C₁₋₃ amine, NH, O, sulfo and sulfhydryl residues.

Preferably, at least one of R₁, R₂, R₃, R₄, R₅, R₆, R₁₀ and/or R₁, represents a linking residue to the surface of the substrate. The linking residue is, preferably, covalently linked to the surface of the substrate. In some embodiments the linking residue, preferably R₆, R₁₀ and/or R₁₁, may represent a derivative of a functional group (such as an amine group) provided on the surface of the substrate for the purpose of linking a compound according to Formula (II) or (III). Such functional groups may, in turn, have been provided on the substrate surface via a binding intermediary (eg an intermediate layer).

Preferably, the substrate comprises less than about 10 mg of the compound per cm² of substrate surface.

In a second aspect, the invention provides for a device comprising a substrate according to the first aspect.

The device is preferably a medical device, however it may otherwise be a device for which the control or inhibition of microbial infection or colonisation is desirable such as, for example, a female hygiene product or a food cooking, preparation or storage utensil or container. In each case, the nature and properties of the substrate may be selected for particular suitability for the intended purpose, though, preferably, the substrate comprises a thin film perfluorinated poly(ethylene-co-propylene)polymer (FEP).

In a third aspect, the present invention provides a method for immobilising a compound according to Formula (I) to at least one surface of a substrate, said method comprising the steps of;

(i) optionally preparing the surface of the substrate to be reactive with the compound; and
(ii) reacting the compound with the surface so as to immobilise the compound onto the surface.

In a preferred method according to the third aspect, step (i) comprises coating the surface of the substrate with an intermediate layer comprising surface functional groups, wherein the intermediate layer may be formed by deposition of a thin plasma polymer layer, and step (ii) may comprise covalently immobilising the compound to the surface of the substrate.

Alternatively, a compound according to Formula (I) may be immobilised to a surface of a substrate in a single step by thin plasma polymer deposition.

Thus, in a fourth aspect, the present invention provides a method for immobilising a compound according to Formula (I) to at least one surface of a substrate, said method comprising covalently immobilising the compound to said surface by thin plasma polymer deposition.

In a fifth aspect, the present invention provides a compound according to Formula (IV):

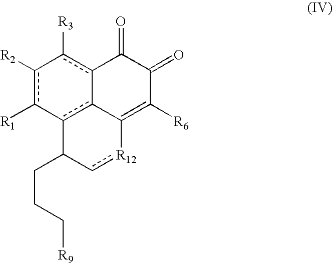

wherein
R₁, R₂, R₃ and R₆ are each independently selected from the group consisting of hydrogen, halogen, hydroxyl, formyl, alkyl, alkoxy, oxoalkyl, alkenyl, aryl, arylalkyl, carbonyl, carboxyl, aldehyde, carboxylate, cyano, ester, ether, carboxamide, amide, amine, sulfo, and sulfhydryl residues, their acids or salts, or derivatives thereof,
R₉ and R₁₂ are each independently selected from the group consisting of C₁₋₃ hydroxyl, C₁₋₃ alkyl, C₁₋₃ alkoxy, C₁₋₃ oxoalkyl, C₁₋₃ alkenyl, C₁₋₃ carbonyl, C₁₋₃ carboxyl, C₁₋₃ aldehyde, C₁₋₃ carboxylate, C₁₋₃ cyano, C₁₋₃ ester, C₁₋₃ ether, C₁₋₃ carboxamide, C₁₋₃ amide, C₁₋₃ amine, NH, O, sulfo and sulfhydryl residues, or derivatives thereof, and
wherein said compound is other than naphtho(1,8-bc)pyran-7,8-dione (ie biflorin).

Preferably, R₉ is:

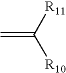

wherein
R₁₀ and R₁₁ are independently selected from the group consisting of hydrogen, halogen, hydroxyl, formyl, alkyl, alkoxy, oxoalkyl, alkenyl, aryl, arylalkyl, carbonyl, carboxyl, aldehyde, carboxylate, cyano, ester, ether, carboxamide, amide, amine, sulfo, and sulfhydryl residues, their acids or salts, or derivatives thereof.

### BRIEF DESCRIPTION OF THE FIGURES

FIG. 1 shows a diagrammatic representation of the bicarbocyclic structure of serrulatanes, compound (1), and the chemical structures of compounds (2) to (8), as described herein, isolated from Eremophila extracts.

FIG. 2 shows the multiple-bond CH correlation (HMBC) of the aromatic moiety of compounds (2) and (3) described herein.

FIG. 3 (a) provides a diagrammatic illustration of each step involved in the immobilisation via oxymercuration of a serrulatane compound to the surface of a flat sheet of perfluorinated poly(ethylene-co-propylene)polymer (FEP), and (b) provides a diagrammatic illustration of the steps involved in the covalent immobilisation of carboxylated serrulatanes via carbodiimide-mediated amide bond formation.

FIG. 4 shows an X-ray photoelectron spectroscopy (XPS) spectrum recorded on a sample after immobilisation of a serrulatane compound onto a flat sheet of FEP via propionaldehyde plasma polymer and a polyallylamine intermediate layer.

FIG. 5 shows a Time-of-Flight Secondary Ion Mass spectrum recorded on a sample after immobilisation of a serrulatane via amide bond formation. Characteristic mass signals assignable to serrulatane-derived ions indicate the presence of the compound on the surface, and polyallylamine-derived ions show the presence of a polyallylamine intermediate layer.

FIG. 6 provides stained photomicrographs of bacterial colonisation of polyallylamine (left hand panel) and polyallylamine+serrulatane (centre and right hand panels) surfaces. The serrulatane compound was linked to the surface via oxymercuration (centre panel) or via carbodiimide catalysis (right hand panel).

FIG. 7 shows photographs of cell culture wells to which bacterial suspensions had been added and biofilm growth allowed to proceed for 24 hours. Samples were stained with a 0.1% safranin solution for the presence of biofilm. The samples are (from left) a polyallylamine control and serrulatane coatings comprising compound 8 (ie 8-hydroxyserrulat-14-en-19-oic acid) immobolised at four different concentrations, 0.06 M, 0.03 M, 0.015 M and 0.0075 M.

FIG. 8 shows photomicrographs of the colonisation of the surfaces of samples by mouse 3T3 fibroblast cells. The samples were a polyallylamine control and two serrulatane coatings comprising compound 8 (ie 8-hydroxyserrulat-14-en-19-oic acid) immobolised at 0.015 M and 0.0075 M concentrations. The morphology of the cells indicates good cell compatibility.

FIG. 9 shows the number of cells per mm² for the sample surfaces shown in FIG. 8, obtained by statistical image analysis of several fields of view for each sample.

### DETAILED DESCRIPTION OF THE INVENTION

The present invention is described herein with particular reference to medical device applications. However, it is to be understood that the present invention has applications in any area wherein the control or inhibition of microbial infection or colonisation, particularly bacterial infection or colonisation, is desirable. Examples of medical devices that can be prone to bacterial colonisation include implantable medical devices such as replacement joints (eg knee and hip joints), urinary catheters, percutaneous access catheters, stents, as well as non-implantable devices such as contact lenses and masks and apparatus for breathing medical air or oxygen. Examples of other surfaces which desirably are kept free of bacterial colonisation include contact lens storage cases, food cooking and preparation surfaces, and the like. The present invention also extends to novel compounds which exhibit antimicrobial activity. Such compounds may be suitable for various applications wherein the control or inhibition of microbial infection or colonisation is desirable, but may find particular utility in the prevention of bacterial infection or colonisation of solid surfaces.

In a first aspect, the present invention provides a substrate comprising at least one surface wherein said surface comprises a compound according to Formula (I) immobilised thereto:

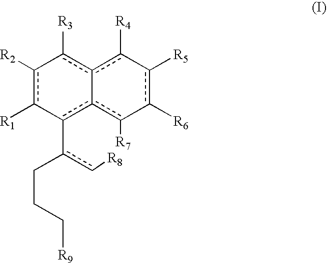

wherein
R₁, R₂, R₃, R₄, R₅, R₆ and R₉ are each independently selected from the group consisting of hydrogen, halogen, hydroxyl, formyl, alkyl, alkoxy, oxoalkyl, alkenyl, aryl, arylalkyl, carbonyl, carboxyl, aldehyde, carboxylate, cyano, ester, ether, carboxamide, amide, amine, sulfo, and sulfhydryl residues, their acids or salts, or derivatives thereof; and
R₇ and R₈ are each independently selected from the group consisting of hydrogen, halogen, hydroxyl, formyl, alkyl, alkoxy, oxoalkyl, alkenyl, aryl, alkylaryl, carbonyl, carboxyl, aldehyde, carboxylate, cyano, ester, ether, carboxamide, amide, amine, sulfo, and sulfhydryl residues, their acids or salts, or derivatives thereof, or together form a C₁₋₃ or heteroatom bridge to form a cyclic or heterocyclic group.

In the Formula (I), it is to be understood that:

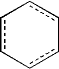

indicates that the ring structures can have, independently, 0 to 3 double bonds (ie C═C) at any ring position and may include a resonance structure. Similarly, [symbol] is to be understood as representing either a single bond or a double bond.

Preferably, R₉ is selected from the group consisting of C₁₋₃ hydroxyl, C₁₋₃ alkyl, C₁₋₃ alkoxy, C₁₋₃ oxoalkyl, C₁₋₃ alkenyl, C₁₋₃ carbonyl, C₁₋₃ carboxyl, C₁₋₃ aldehyde, C₁₋₃ carboxylate, C₁₋₃ cyano, C₁₋₃ ester, C₁₋₃ ether, C₁₋₃ carboxamide, C₁₋₃ amide, C₁₋₃ amine, NH, O, sulfo and sulfhydryl residues, or derivatives thereof. More preferably, R₉ is selected from C₁₋₃ hydroxyl, C₁₋₃ alkyl, C₁₋₃ alkoxy, C₁₋₃ carboxyl, C₁₋₃ amine and C₁₋₃ alkenyl, or derivatives thereof.

Most preferably, R₉ is:

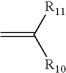

wherein
R₁₀ and R₁₁ are independently selected from the group consisting of hydrogen, halogen, hydroxyl, formyl, alkyl, alkoxy, oxoalkyl, alkenyl, aryl, arylalkyl, carbonyl, carboxyl, aldehyde, carboxylate, cyano, ester, ether, carboxamide, amide, amine, sulfo, and sulfhydryl residues, their acids or salts, or derivatives thereof.

At least one of R₁, R₂, R₃, R₄, R₅, R₆, R₉, R₁₀ and R₁₁ represents a linking residue to the surface of the substrate, preferably by a covalent linkage. More preferably, at least one of R₆, R₉, R₁₀ and R₁₁ represent a linking residue. Suitable linking residues include, for example, amine, acetyl, hydroxyl, carboxyl, alkenyl, and other reactive groups known in the art may act as linking residues. Further, acetyl groups (—O—COCH₃) may be hydrolysed to hydroxyl groups to provide a reactive group at a desired position. In some embodiments, the linking residue may represent a derivative of a functional group (such as an amine group) provided on the surface of the substrate for the purpose of linking a compound according to Formula (I). Such functional groups may, in turn, have been provided on the substrate surface via a binding intermediary (eg an intermediate layer).

R₇ and R₈ are less suitable for forming linking residues due to steric constraints.

Preferably, the compound is according to Formula (II) or (III):

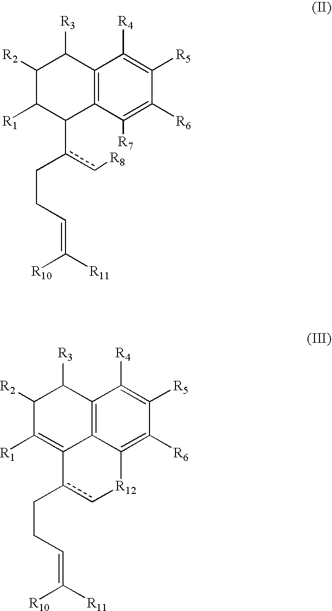

wherein
R₁, R₂, R₃, R₄, R₅, R₆, R₇, R₈, R₁₀ and R₁₁ are each independently selected from the group consisting of hydrogen, halogen, hydroxyl, formyl, alkyl, alkoxy, oxoalkyl, alkenyl, aryl, arylalkyl, carbonyl, carboxyl, aldehyde, carboxylate, cyano, ester, ether, carboxamide, amide, amine, sulfo, and sulfhydryl residues, their acids or salts, or derivatives thereof; and
R₁₂ is selected from the group consisting of C₁₋₃ alkyl, C₁₋₃ alkoxy, C₁₋₃ oxoalkyl, C₁₋₃ alkenyl, C₁₋₃ carbonyl, C₁₋₃ carboxyl, C₁₋₃ aldehyde, C₁₋₃ carboxylate, C₁₋₃ cyano, C₁₋₃ ester, C₁₋₃ ether, C₁₋₃ carboxamide, C₁₋₃ amide, C₁₋₃ amine, NH, O, sulfo and sulfhydryl residues, or derivatives thereof.

In the Formula (III), it is to be understood that [symbol] represents either a single bond or a double bond.

For R₁, R₂, R₃, R₄, R₅, R₆, R₇, R₈, R₁₀ and R₁₁, preferred alkyl residues of compounds according to the first aspect include straight chain or branched C₁₋₆ alkyl groups, for example, methyl, ethyl, propyl and isopropyl residues. Preferred alkoxy residues include C₁₋₆ straight chain or branched alkoxy such as methoxy, ethoxy, n-propoxy, isopropoxy and butoxy isomers. Preferred alkenyl residues include C₁₋₆ straight chain, branched or mono- or polycyclic alkenes including ethylenically mono- or poly-unsaturated alkyl or cycloalkyl groups (eg vinyl, allyl, 1-methylvinyl, butenyl, iso-butenyl, 1-pentenyl, cyclopentenyl, 1-methyl-cyclopentenyl, 1-hexenyl, 3-hexenyl, cyclohexenyl, 1-heptenyl and 3-heptenyl).

Preferred halogen residues include fluorine, chlorine, bromine and iodine. Preferred acyl residues include C₁₋₆ carbamoyls, aliphatic acyl groups and acyl groups containing a heterocyclic ring (referred to as heterocyclic acyl), straight chain or branched alkanoyl, such as formyl, acetyl, propanoyl, butanoyl, 2-methylpropanoyl, pentanoyl, and hexanoyl).

R₁ and R₅ are most preferably hydrogen.
R₂ is most preferably hydrogen, hydroxyl, or —O(COCH₃).
R₃ is most preferably hydrogen, methyl, —CH₂OH, carboxyl, or —CH₂O(COCH₃).
R₄ is most preferably hydrogen, hydroxyl, carbonyl or —O(COCH₃).
R₆ is most preferably methyl, carboxyl, —CH₂OH, or CH₂O(COCH₃).
R₇ and R₈, most preferably, form together an oxygen atom bridge to thereby form a heterocyclic ring.
R₁₀ and R₁₁ are most preferably methyl or —CH₂OH.

Particularly preferred compounds include 9-methyl-3-(4-methyl-3-pentenyl)-2,3-dihydronaphtho[1,8-bc]pyran-7,8-dione, a serrulatane-type diterpenoid, 20-acetoxy-8-hydroxyserrulat-14-en-19-oic acid, 8,20-dihydroxyserrulat-14-en-19-oic acid, 8,20-diacetoxyserrulat-14-en-19-oic acid, serrulatane-type diterpenoids, 2,19-diacetoxy-8-hydroxyserrulat-14-ene, 8,19-dihydroxyserrulat-14-ene, and 8-hydroxyserrulat-14-en-19-oic acid, serrulat-14-en-7,8,20-triol, serrulat-14-en-3,7,8,20-tetraol, their acids or salts, or derivatives thereof.

The substrate may comprise less than about 10 mg of the compound per cm² of substrate surface, more preferably about 0.001 μg to 10 mg. Preferably, many molecules of the compound are provided such as, for example, may be sufficient to form a film or coating on the substrate. Indeed, the compound may be provided in excess such that a confluent coating across the surface of the substrate is achieved. Alternatively, the compound may be uniformly distributed but provide less than confluent coverage across the surface of the substrate, for example, where the compound has particularly high antimicrobial activity.

In a particularly preferred embodiment of the present invention, the compound is covalently linked via an amine group provided by a layer of polyallylamine, which is, in turn, covalently bound to an intermediate aldehyde plasma polymer layer on the substrate surface.

Compounds according to Formula (I) may be derived from naturally occurring sources, for example from Eremophila plants, or they may be synthetically generated, for example as described in Dehmel F et al. (2002) or Ng PSP and Benerjee A K (2006), or synthetically generated from naturally occurring precursors, for example, the hydrolysis of serrulat-14-en-7,8,20-triol and serrulat-14-en-3,7,8,20-tetraol to their more active derivatives.

It will be understood that more than one type of compound according to the Formula (I) may be immobilised to the substrate surface (eg a single variety of compound or plurality of different compounds selected, for example, for their antibacterial activity and/or absence of cytotoxicity, may be immobilised on the substrate surface).

The antimicrobial activity of compounds according to Formula (I) is discussed in the examples provided hereinafter. Further, it is considered that at least some of compounds according to Formula (I) will additionally possess anti-inflammatory activity (Liu Q et al., 2006). That is, it has been previously found that serrulatic acid isolated from a crude ethanol extract of E. sturti, inhibited cyclooxygenase 1 (COX-1) and cyclooxygenase 2 (COX-2), with 95% and 89% inhibition of COX-1 and COX-2, respectively, at 2 mg/ml (Liu Q et al., 2006). Accordingly, the substrate of the present invention may also provide benefits in applications where the control or reduction of inflammation is desirable.

The material from which the substrate is composed will preferably be solid, however gels and other “soft” or semi-solid materials may also be suitable. Typically, the substrate material will be selected to suit a particular intended application. For example, in the case of a shaped medical device, the material may be selected to meet certain specifications of a particular intended application, such as mechanical and optical properties.

A solid substrate according to the present invention may be shaped or non-shaped, rigid or flexible. Moreover, a solid substrate according to the present invention may be a coating or woven or non-woven film or sheet. Alternatively, the solid substrate may consist of micro- or nano-sized particles.

The solid substrate may be composed from a natural or synthetic filament or fibre (eg a plant material). Alternatively, the solid substrate may be composed from a metal, a ceramic, a solid synthetic polymer, or a solid natural polymer, for example a solid biopolymer. Examples of preferred materials include titanium, hydroxyapatite, polyethylene (which are useful for orthopaedic implants), polyurethanes, organosiloxane polymers, perfluorinated polymers (which are useful for catheters, soft tissue augmentation implants, and blood contacting devices such as heart valves), acrylic hydrogel polymers and siloxane hydrogel polymers (eg for contact lens and intraocular lens applications), and the like, and any combination thereof.

A number of coating methods are suitable for the preparation of solid substrates according to the present invention, for example, thin film perfluorinated poly(ethylene-co-propylene)polymer (FEP) is suitable for coating a range of solid substrates. Accordingly, thin film FEP coated with immobilised antimicrobial compounds may be used as a coating for medical devices, including replacement joints, catheters and stents.

In a second aspect, the invention provides for a device comprising a substrate according to the first aspect.

The device is preferably a medical device, however it may otherwise be a device for which the control or inhibition of microbial infection or colonisation is desirable such as, for example, a female hygiene product or a food cooking, preparation or storage utensil or container. In each case, the nature and properties of the substrate may be selected for particular suitability for the intended application.

The term “medical device” as used herein is intended to have a broad definition and may include any apparatus for permanent or temporary use on or in a body, for use in human or veterinary applications, or associated apparatus, for example vessels for cleaning or storing such apparatus.

Preferred medical devices include implantable and non-implantable devices, for example, replacement joints, urinary catheters, percutaneous access catheters, stents, as well as non-implantable devices such as contact lenses and masks and apparatus for breathing medical air and oxygen.

In a third aspect, the present invention provides a method for immobilising a compound according to Formula (I) to at least one surface of a substrate, said method comprising the steps of;

(i) optionally preparing the surface of the substrate to be reactive with the compound; and
(ii) reacting the compound with the surface so as to immobilise the compound onto the surface.

The specific methods selected for steps (i) and (ii) may vary depending upon, for example, the surface properties of the substrate and/or the intended application of the substrate. The modification of the method steps to adjust to different substrates includes modifications well known to persons skilled in the art. These may require adjustment by routine methods for the optimisation of the method.

Methods for preparing the surface may include processes for the cleaning of the surface, for example washing, autoclaving, irradiating and/or coating the surface, and/or processes for providing reactive functional groups to the substrate surface and/or attaching a binding intermediary (eg an intermediate layer) to the substrate surface. Alternatively, substrates may be manufactured with the necessary surface characteristics required for reacting the compound with the surface, thus, the surface preparation step may not be required; it merely being necessary that there is provided a surface of the substrate that is reactive with the compound.

Suitable methods for coating the surface of the substrate and/or for the preparation of an intermediate layer comprising surface functional (ie reactive) groups are well known to persons skilled in the art. Such methods may be undertaken in a single step or, alternatively, in multiple steps, usually comprising a coating step and a binding step for attaching a functional group. One such suitable method comprises plasma polymerisation of a suitable material (eg propionaldehyde) to form a coating on the surface of the substrate followed by the covalent binding of a functional group (eg an amine group) thereto. Such methods are known to be broadly applicable to many types of substrate surfaces. An alternative method, comprising the deposition of a thin plasma polymer layer, may involve a single coating and binding step. As mentioned above, the compound is preferably covalently bound to the surface of the substrate through, for example, an oxymercuration or an amide forming reaction. Suitable binding intermediate layers for undertaking both oxymercuration and amide formation reactions include those with a surface comprising amine groups.

In a fourth aspect, the present invention provides a method for immobilising a compound according to Formula (I) to at least one surface of a substrate, said method comprising covalently immobilising the compound to said surface by thin plasma polymer deposition.

Compounds may be immobilised, by the method of the third or fourth aspect, to the substrate surface by the “head”, preferably by residues R₁, R₂, R₃, R₄, R₅, and/or R₆, most preferably by R₆, or by the “tail”, preferably by residues R₉, R₁₀ and/or R₁₁ of the molecule. Covalent binding via the “tail” of the molecule may be preferred to enhance the activity of certain molecules. For example, the binding of quinone compounds to the substrate at the “tail” end of the molecule exposes the active quinone moiety at the “head” of the molecule thereby enhancing activity of the molecule.

Compounds suitable for use in the methods of the present invention may be derived from plant material from Eremophila species, such as E. serrulata and E. neglecta. Methods for the identification of alternative compounds according to Formula (I) are described in the examples provided hereinafter. Compounds suitable for use in the methods of the present invention may be provided by purified or partially purified extracts of Eremophila species. Alternatively, compounds produced by organic chemical synthesis routes are suitable.

Optionally, methods according to the present invention may involve the addition of stabilising agents to stabilise the compound according to Formula (I) prior to, and/or during, and/or following the immobilisation of the compound to a substrate surface. Stabilising agents may include antioxidants, such as vitamin E, and nitrogen gas. Alternatively, a compound according to Formula (I) may be converted to a more stable derivative, for example acetylated derivatives, which may subsequently be hydrolysed following immobilisation to the substrate surface.

Further optional steps in the method of the third or fourth aspect may include a step of moulding the substrate into a desired shape, for example in the desired shape of a cosmetic implant, and/or one or more steps of solubilising, suspending, melting and crushing a compound preparation substance into fine particles for distribution onto the substrate, followed by the setting of a solution, suspension, liquid, or film (eg by drying).

In a fifth aspect, the present invention provides a compound according to Formula (IV):

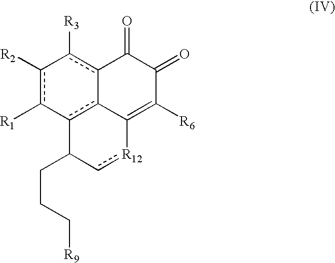

wherein
R₁, R₂, R₃ and R₆ are each independently selected from the group consisting of hydrogen, halogen, hydroxyl, formyl, alkyl, alkoxy, oxoalkyl, alkenyl, aryl arylalkyl, carbonyl, carboxyl, aldehyde, carboxylate, cyano, ester, ether, carboxamide, amide, amine, sulfo, and sulfhydryl residues, their acids or salts, or derivatives thereof;
R₉ and R₁₂ are each independently selected from the group consisting of C₁₋₃ hydroxyl, C₁₋₃ alkyl, C₁₋₃ alkoxy, C₁₋₃ oxoalkyl, alkenyl, C₁₋₃ carbonyl, C₁₋₃ carboxyl, C₁₋₃ aldehyde, C₁₋₃ carboxylate, C₁₋₃ cyano, C₁₋₃ ester, C₁₋₃ ether, C₁₋₃ carboxamide, C₁₋₃ amide, C₁₋₃ amine, NH, O, sulfo and sulfhydryl residues, or derivatives thereof, and
wherein said compound is other than naphtho(1,8-bc)pyran-7,8-dione (ie biflorin).

Preferably, the compound is according to Formula (V):

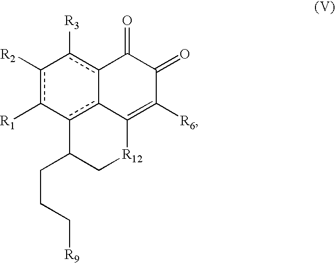

### or Formula (VI):

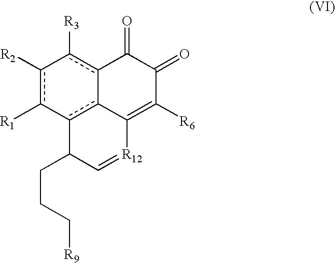

wherein R₁₂ is selected from the group consisting of C₁₋₃ hydroxyl, C₁₋₃ alkyl, C₁₋₃ alkoxy, C₁₋₃ oxoalkyl, C₁₋₃ alkenyl, C₁₋₃ carbonyl, C₁₋₃ carboxyl, C₁₋₃ aldehyde, C₁₋₃ carboxylate, C₁₋₃ cyano, C₁₋₃ ester, C₁₋₃ ether, C₁₋₃ carboxamide, C₁₋₃ amide, C₁₋₃ amine, NH, sulfo and sulfhydryl residues, or derivatives thereof.

Most preferably, R₉ is:

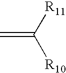

wherein
R₁₀ and R₁₁ are independently selected from the group consisting of hydrogen, halogen, hydroxyl, formyl, alkyl, alkoxy, oxoalkyl, alkenyl, aryl, arylalkyl, carbonyl, carboxyl, aldehyde, carboxylate, cyano, ester, ether, carboxamide, amide, amine, sulfo, and sulfhydryl residues, their acids or salts, or derivatives thereof.

Alternatively, the compound is preferably according to Formula (VII):

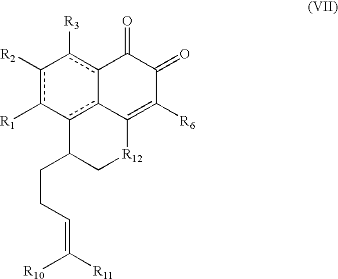

wherein
R₁, R₂, R₃, R₆, R₁₀ and R₁₁ are each independently selected from the group consisting of hydrogen, halogen, hydroxyl, formyl, alkyl, alkoxy, oxoalkyl, alkenyl, aryl, arylalkyl, carbonyl, carboxyl, aldehyde, carboxylate, cyano, ester, ether, carboxamide, amide, amine, sulfo, and sulfhydryl residues, their acids or salts, or derivatives thereof; and
R₁₂ is selected from the group consisting of C₁₋₃ alkyl, C₁₋₃ alkoxy, C₁₋₃ oxoalkyl, C₁₋₃ alkenyl, C₁₋₃ carbonyl, C₁₋₃ carboxyl, C₁₋₃ aldehyde, C₁₋₃ carboxylate, C₁₋₃ cyano, C₁₋₃ ester, C₁₋₃ ether, C₁₋₃ carboxamide, C₁₋₃ amide, C₁₋₃ amine, NH, O, sulfo and sulfhydryl residues, or derivatives thereof.

In Formulae (IV) to (VII), it is to be understood that:

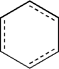

indicates that the ring structure may have 0 to 3 double bonds (ie C═C) at any ring position and may include a resonance structure.

In Formulae (IV) to (VII), R₆, R₉, R₁₀ and R₁₁ are each independently selected from C₁₋₃ hydroxyl, C₁₋₃ alkyl, C₁₋₃ alkoxy, C₁₋₃ carboxyl, C₁₋₃ amine and C₁₋₃ alkenyl, or derivatives thereof.

In one embodiment, the compound of the fifth aspect is also other than 9-methyl-3-(4-methyl-3-pentenyl)-2,3-dihydronaphthol[1,8-bc]-pyran-7,8-dione.

The compound of the fifth aspect may be formulated with, for example, a pharmaceutically- or veterinary-acceptable carrier for the preparation of an antibacterial and/or anti-inflammatory composition for human therapeutic or veterinary application. Such a composition may be formulated for, for example, oral or systemic administration.

The present invention also extends to a substrate incorporating a compound according to Formula (I) therein. As such, the substrate may comprise the compound both at the surface and below the surface. The compound may be bound to the substrate material (eg a metal, a ceramic, or a polymer) or may otherwise be present merely in admixture. The compound may therefore exert antimicrobial activity following “leaching” from the substrate or, otherwise, following degradation of the substrate such that “fresh” compound becomes exposed at the surface.

The present invention is hereinafter further described by way of the following, non-limiting examples and accompanying figures.

### EXAMPLES

### Example 1

### Methods and Materials

### Extraction and Isolation

Fresh plant leaves were soaked in diethyl ether (Analytical grade, Merck Pty Limited, Kilsyth, VIC, Australia) overnight in a closed container to extract leaf resins. The solvent was drained and evaporated to dryness in vacuo at 40° C. to produce 40 g of a dark greenish residue, which was re-dissolved in a suitable solvent for further extraction and fractionation.

### Antimicrobial Assays

a. Bacterial Strains and Media

Staphylococcus aureus ATCC 29213 obtained from stock cultures stored at −70° C. at the Sansom Institute (University of South Australia, Adelaide, SA, Australia) was used as the test microorganism in a bioassay guided fractionation process. S. aureus ATCC 25923, Streptococcus pyogenes ATCC 10389, Streptococcus pneumoniae ATCC 49619, Salmonella typhimurium ATCC 13311, Pseudomonas aeruginosa ATCC 27853, and Escherichia coli ATCC 25922, from the same collection were used to test the most active compound isolated. All bacteria were grown on blood agar plates (Colombia agar-CM331, Oxoid Limited, Basingstoke, Hampshire, United Kingdom) supplemented with 5% v/v sheep blood) at 37° C. The Streptococcus species were incubated at 37° C. in the presence of 5% carbon dioxide (CO₂). Brain Heart Infusion broth (CM 225, Oxoid Limited) was used for the experiments to determine the minimum inhibitory concentration (MIC) and minimum bactericidal concentration (MBC) for the Streptococcus species while cation adjusted MH II broth (Becton Dickinson France SAS, Le Point de Claix, France) was used for the Staphylococcus species and the Gram-negative bacteria tested.

b. Broth Micro Dilution Assay for Determination of MIC and MBC

Broth micro dilution was used to determine the MIC of extract, fractions and pure compounds against S. aureus ATCC 29213. Duplicate two-fold serial dilutions of test sample (100 μL/well) were prepared in sterile round-bottom 96-well plates (Sarstedt Australia Pty Ltd, Technology Park, SA, Australia), in the appropriate broth containing 2% DMSO. Bacterial cell suspension (100 μL) corresponding to 1×10⁶ CFU/mL was added in all wells with the exception of wells containing saline, test sample and media sterility controls, respectively. Controls for bacterial growth without test sample were also included on each plate. Plates were then placed on a shaker for 10 min and incubated at 37° C. overnight. After incubation, the MIC was determined as the lowest concentration at which no growth was observed in the duplicate wells. Vancomycin and gentamicin (Sigma, St Louis, Mo., United States of America) were used as positive controls for the Gram-positive and Gram-negative bacteria, respectively. Following the determination of the MIC, the MBC was determined by transferring a 10 μL aliquot from each of the wells at the concentration corresponding to the MIC and those concentrations above into 190 μL of appropriate broth in a sterile 96-well plate. The plates were incubated under the same conditions as in the MIC experiment with the Streptococcus species incubated in the presence of 5% CO₂. The presence or absence of bacterial growth was determined by visual inspection. The MBC was considered to be the lowest concentration of the compound at which no growth occurred.

### General Experimental Procedures

Merck Si gel 60 (70-230 mesh ASTM) and Sephadex LH-20 (Sigma) were used for column chromatography. High Performance Liquid Chromatography (HPLC) experiments were performed on a Shimadzu LC-6A system with Activon Goldpak C-18 reversed-phase and normal-phase (SiO2) semipreparative (25×1 cm) HPLC columns.

Melting points were determined using a Stuart Scientific SMP10 apparatus. Optical rotations were measured on an Atago AP100 polarimeter. Ultra violet (UV) spectra were recorded on a Shimadzu UV-1700 Pharma Spec spectrophotometer. Infrared (IR) spectra were measured on an FT-IR-8400 S Shimadzu spectrometer. 1D and 2D Nuclear Magnetic Resonance (NMR) spectra were acquired on a Varian INOVA 600 MHz spectrometer. Chemical shifts were referenced to residual solvent resonances. High- and low-resolution mass spectra were obtained on a Kratos Concept ISQ magnetic sector mass spectrometer.

### Results and Discussion

Bioactive Compounds from E. serrulata

a. Plant Material of E. serrulata

Leaves of E. serrulata were collected in northern South Australia, 135.2 km north of Glendambo.

b. Isolation and Identification of Bioactive Compounds

E. serrulata leaf extract was re-dissolved in CH₂Cl₂ and washed sequentially with 8% w/v aqueous NaHCO₃ and 5% w/v aqueous NaOH solution as described previously (Forster P G et al., 1986). The aqueous basic fractions were acidified with 10% H₂SO₄, re-extracted with CH₂Cl₂ and filtered through activated charcoal. The different CH₂Cl₂ portions were dried using anhydrous Na₂SO₄, filtered and evaporated to yield NaHCO₃-soluble (MIC=1000 μg/mL), NaOH-soluble (MIC=125 μg/mL) and neutral CH₂Cl₂ (MIC=125 μg/mL) fractions, respectively.

The diethyl ether extract of E. serrulata exhibited antimicrobial activity against S. aureus ATCC 29213 with a MIC of 125 μg/mL. The NaHCO₃ soluble, NaOH soluble and neutral CH₂Cl₂ fractions obtained by partitioning the crude diethyl ether extract were also examined for antimicrobial activity. Both the NaOH soluble and the neutral fractions were found to be the most active with each having a MIC of 125 μg/mL.

TABLE 1
Antimicrobial activity of compounds 2-5 against S. aureus
ATCC 29213

| | Antimicrobial activity |
|---|---|
| Compound | MIC (μg/mL) |
| 2 | 15.6 |
| 3 | 125 |
| 4 | 250 |
| 5 | 250 |

The NaOH soluble fraction (11 g) was subjected to vacuum liquid chromatography eluting with CH₂Cl₂ and increasing amounts of ethyl acetate. Eighteen fractions were collected and grouped based on their thin layer chromatography (TLC) profile into four major fractions (F1-F4).

Fraction F1 eluted from the main column with CH₂Cl₂ formed a powder when dissolved in chloroform. This was filtered to afford a yellow powder (MIC=500 μg/mL) and a filtrate (MIC=125 μg/mL). The filtrate (225 mg) was passed through a Sephadex column eluting with CH₂Cl₂/MeOH 3:1 to afford 32 fractions, which were grouped into three major fractions (F1-1, F1-2 and F1-3).

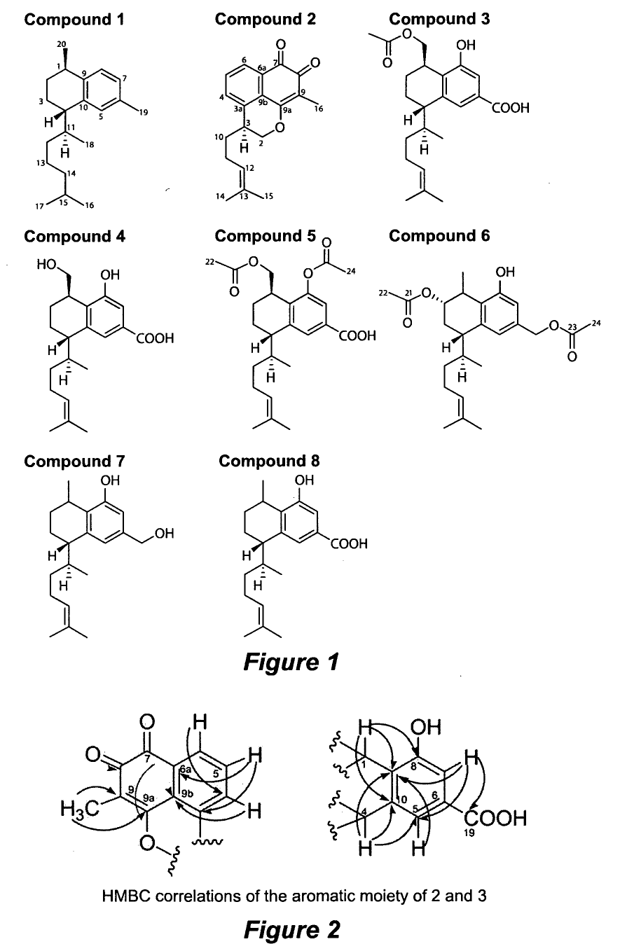

Fraction F1-1 (MIC=15.6 μg/mL) was further separated isocratically by reverse phase (RP)-HPLC using 75% MeOH/water with 0.1% formic acid as eluent and a flow rate of 2 mL/min. Seventy-two fractions were collected and grouped into eight different fractions (F1-1-1 to F1-1-8). Fraction F1-1-4 afforded 10 mg of pure compound (2) (FIG. 1) (MIC=15.6 μg/mL) as an amorphous orange solid.

Fraction F3 (500 mg, MIC=250 μg/mL) eluted from the vacuum liquid chromatography column with CH₂Cl₂/MeOH 1:1 was passed through a reverse phase HPLC column using 65% MeOH/water with 0.1% formic acid as eluent and a flow rate of 2 mL/min. Seventy fractions were collected and grouped into three main fractions (F3-1 to F3-3). F3-3 afforded 20 mg of 8,20-dihydroxyserrulat-14-en-19-oic acid (compound (4); FIG. 1) (MIC=250 μg/mL) as a pale yellowish oil.

Treatment of 2.5 g of the neutral fraction (MIC=125 μg/mL) by vacuum liquid chromatography eluting with CH₂Cl₂ and increasing amounts of ethyl acetate afforded 22 fractions which were grouped into six major fractions (ESAF1-6) based on their TLC profile.

Fraction ESAF4 which came out from the main column with CH₂Cl₂/EtOAc 1:1 was further separated by RP-HPLC using 75% MeOH/water with 0.1% formic acid as eluent and a flow rate of 2 mL/min. Fifty fractions were collected and grouped into four different fractions (ESAF4-1 to ESAF4-4). ESAF4-2 (MIC=125 μg/mL) was passed through a Sephadex column eluting with CH₂Cl₂/MeOH 3:1 to afford 35 fractions, which were grouped into three fractions (ESAF4-2-1 to ESAF4-2-3). ESAF4-2-3 afforded 25 mg of compound (3) (FIG. 1) (MIC=125 μg/mL) as a pale yellowish oil.

Similarly, fraction ESAF4-4 afforded 30 mg 8,20-diacetoxyserrulat-14-en-19-oic acid (compound (5); FIG. 1) (MIC=250 μg/mL) as a pale yellowish oil after it was passed through a Sephadex column and eluted with CH₂Cl₂/MeOH 3:1.

The Rf values for compounds (2), (3), (4) and (5) on normal phase TLC (Merck, Silica gel 60, F254, Darmstadt, Germany) when methylene chloride:methanol (9:1) was used as the mobile phase were 0.750, 0.388, 0.350, and 0.488, respectively. These spots were also present in the original diethyl ether extract before the partitioning with the bases.

### a. 9-Methyl-3-(4-methyl-3-pentenyl)-2,3-dihydronaphtho[1,8-bc]-pyran-7,8-dione and 20-Acetoxy-8-hydroxyserrulat-14-en-19-oic acid

Bioassay guided fractionation of the NaOH soluble and the neutral CH₂Cl₂ fractions led to the isolation of two new compounds, 9-methyl-3-(4-methyl-3-pentenyl)-2,3-dihydronaphtho[1,8-bc]pyran-7,8-dione (compound (2)) and a serrulatane diterpenoid, 20-acetoxy-8-hydroxyserrulat-14-en-19-oic acid (compound (3)) together with two known (Forster P G et al., 1986) serrulatane-type diterpenoids, 8,20-dihydroxyserrulat-14-en-19-oic acid (compound (4)) and 8,20-diacetoxyserrulat-14-en-19-oic acid (compound (5)). The numbering system for compound (2) follows that for the previously described related compound named biflorin (Fonseca A M et al., 2003).

Both of the new compounds were active against S. aureus ATCC 29213 with compound (2), showing an MIC of 15.6 μg/mL (Table 1). The structures of these compounds were elucidated using one- and two-dimensional NMR, IR and UV/visible spectroscopy and mass spectrometry. Compounds (4) and (5) were identified by comparing their NMR and mass spectral data with reported values (Forster P G et al., 1986). Compounds (2)-(5) showed antimicrobial activity against S. aureus (ATCC 29213) with minimum inhibitory concentrations (MICs) ranging from 15.6 to 250 mL (Table 1). Compound (2) exhibited a minimum bactericidal concentration (MBC) of 125 μg/mL. This compound further showed antimicrobial activity against other Gram-positive bacteria including S. aureus (ATCC 25923), S. pyogenes and S. pneumoniae. The MICs and the MBCs ranged from 7.8 to 15.6 μg/mL and 7.8 to 125 μg/mL, respectively (see Table 2).

TABLE 2
Antimicrobial activity of compound 2 against six different
microorganisms

| | Antimicrobial activity | |
|---|---|---|
| Microorganisms | MIC (μg/mL) | MBC (μg/mL) |
| Staphylococcus aureus ATCC 29213 | 15.6 | 125 |
| Staphylococcus aureus ATCC 25923 | 7.8 | 15.6 |
| Streptococcus pneumoniae ATCC | 7.8 | 7.8 |
| 49619 | | |
| Streptococcus pyogenes ATCC 10389 | 7.8 | 15.6 |
| Escherichia coli ATCC 25922 | NA | NA |
| Salmonella typhimurium ATCC 13311 | NA | NA |
| Pseudomonas aeruginosa ATCC 27853 | NA | NA |

NA means not active at maximum concentration tested (250 μg/mL)

Activity was not observed for compound (2) against all Gram-negative bacteria tested. This, together with the recent isolation of two new antimicrobial serrulatane diterpenoids (serrulatic acids) from E. sturtii (Liu Q et al., 2006), confirm previous findings that Eremophila extracts are only active against Gram-positive organisms (Palombo E A and Semple S J, 2001; Shah A et al., 2004; Ndi C P et al., 2007).

9-Methyl-3-(4-methyl-3-pentenyl)-2,3-dihydronaphtho[1,8-bc]-pyran-7,8-dione (compound (2)) is an orange amorphous solid; [α]_{D}²⁴93° (MeOH; c 0.150); λ ₘₐₓ^{CHCl}^{₃} nm (log ε): 268 (4.3), 344 (3.0), 452 (3.3); μₘₐₓ(CHCl₃) cm⁻¹: 1706, 1640, 1615, 1575; ¹H and ¹³C NMR data are shown in Table 3; HREIMS I: 296.1412 [M]⁺, LREIMS m/z: 298 [M+2]⁺, 268, 225. The molecular formula was determined to be C₁₉H₂O₃ from the molecular ion peak at m/z 296.1412 (Calcd. 296.1413) in the high-resolution EI mass spectrum (HREIMS), indicating 10 degrees of unsaturation. The UV spectrum in CHCl₃ showed absorptions at μₘₐₓ 268, 344, and 452 nm characteristic of a typical o-naphthoquinone (Thomson, 1971) and the IR spectrum showed peaks at 1706 and 1640 cm⁻¹ in agreement with the presence of two conjugated carbonyl groups. This was further supported by the presence of the [M+2]⁺ion peak in the EI mass spectra which is characteristic of an ortho-naphthoquinone but not a para-naphthoquinone (Oliver R W A and Rashman R M, 1971).

The ¹³C and the APT NMR spectra indicated a total of 19 carbons (Table 3) with three being methyls (δ_{C} 8.2, 18.3, and 26.3), three methylenes with one directly attached to an oxygen atom (δ_{C} 26.9, 34.6, and 71.9), 4 sp² methines (δ_{C}=125.2, 129.0, 132.5, and 136.7), 1 sp³ methine (δ_{C} 37.9), 6 quaternary sp² carbons (δ_{C} 117.6, 127.7, 131.4, 134.1, 140.4, and 165.4) and two carbonyls 181.5, and 181.7. In total, the 10 sp² carbons (five double bond functionalities) and the two carbonyls accounted for seven degrees of unsaturation. This suggested that the compound contained three rings. Amongst the signals in the NMR spectrum were those for three methyl singlets (δH 1.61, 1.68, and 1.91), a tri-substituted vinyl group with the olefinic proton resonating at δH 5.14 (t, J=6.0 Hz), one benzylic proton (δH 2.99, m), two oxy-methylene protons δH 4.62, dd, J=11.1, 2.1 Hz; δH 4.40, dd, J=11.1, 3.3 Hz, and three aromatic protons (δ_{H} 7.51, m; 7.56, m; and δH 7.89, d, J=6.6 Hz). In the COSY spectrum, correlations were observed between the aromatic protons at δ_{H} 7.89 and 7.56 ppm indicating that they were adjacent to each other and also between the aromatic protons at δ_{H} 7.56 and 7.51 ppm. The benzylic proton showed couplings with the oxy-methylene protons and the protons of a methylene group at δ_{H} 1.78 and 1.69 in the COSY spectrum. This benzylic proton, exhibited HMBC correlations with the quaternary aromatic carbons at δ_{C} 127.7 and 140.4 as well as with the aromatic sp² methine carbon at δ_{C} 136.7 (δ_{H} 7.51, m) while the oxy-methylene protons showed HMBC correlation with the methylene carbon at δ_{C} 34.6, the quaternary aromatic carbon at δ_{C} 140.4 and the quaternary sp² carbon at δ_{C} 165.4 ppm thus indicating the presence of a pyran ring fused to the aromatic ring. HMBC correlations of the other aromatic protons are shown in FIG. 2. The methylene protons at δ_{H} 2.12 ppm which coupled with the olefinic proton (δ_{H} 5.14) and the methylene protons at δ_{H} 1.78 and 1.69 in the COSY spectrum, showed HMBC correlation with the benzylic carbon at δ_{C} 37.9 (δ_{H} 2.99, m). This indicated that the tri-substituted vinylic group was part of a side chain attached to the benzylic carbon through the methylene carbon at δ_{H} 34.6. COSY correlation between the protons of this methylene carbon and the benzylic proton further supported the attachment of the side chain at this location.

TABLE 3
NMR data for compounds 2 and 3

| | 2 | | | 3 | |
|---|---|---|---|---|---|
| CH | δ¹³C | δ¹H (J in Hz) | CH | δ¹³C | δ¹H (J in Hz) |
| 2 | 71.9 | 4.62, dd (11.1, 2.1)4.40, | 1 | 34.0 | 3.42, m |
| | | dd (11.1, 3.3) | 2 | 23.1 | 1.84, m |
| 3 | 37.9 | 2.99, m | 3 | 21.0 | 1.83, 1.73, m |
| 3a | 140.4 | — | 4 | 44.2 | 2.66, m |
| 4 | 136.7 | 7.51, m | 5 | 123.6 | 7.39, br s |
| 5 | 132.5 | 7.56, m | 6 | 130.8 | — |
| 6 | 129.0 | 7.89, d (6.6) | 7 | 114.2 | 7.25, d (1.8) |
| 6a | 131.4 | — | 8 | 156.9 | — |
| 7 | 181.7 | — | 9 | 130.4 | — |
| 8 | 181.5 | — | 10 | 144.2 | — |
| 9 | 117.6 | — | 11 | 39.6 | 1.94, m |
| 9a | 165.4 | — | 12 | 35.0 | 1.26, 1.09, m |
| 9b | 127.7 | — | 13 | 27.7 | 1.98, 1.84, m |
| 10 | 34.6 | 1.78, 1.69, m | 14 | 126.1 | 4.95, t br (7.0) |
| 11 | 26.9 | 2.12, m | 15 | 132.8 | — |
| 12 | 125.2 | 5.14, t (6.0) | 16 | 26.3 | 1.52, br s |
| 13 | 134.1 | — | 17 | 18.2 | 1.62, br s |
| 14 | 18.3 | 1.61, brs | 18 | 19.6 | 0.98, d (6.6) |
| 15 | 26.3 | 1.68, brs | 19 | 171.0 | — |
| 16 | 8.2 | 1.91, s | 20 | 66.9 | 4.32, dd (10.2, 4.0) |
| | | | | | 4.06, t br (10.2) |
| | | | 21 | 173.7 | — |
| | | | 22 | 21.3 | 2.03, s |

Spectra obtained at 600 MHz in CD₃OD.

Cross peaks of the olefinic methine proton (δ_{H} 5.14, t, J=6.0 Hz) with a methylene carbon at δ_{C} 34.6, a quaternary carbon at δ_{C} 134.1, and two methyl carbons at δ_{C} 18.3 and δ_{C} 26.3 were observed in the HMBC spectrum. Other HMBC correlations observed were the correlations from the allylic methyl protons (δ_{H} 1.91) to the quaternary sp² carbons δ_{C} 117.6, δ_{C} 165.4, and the carbonyl at δ_{C} 181.5. The second carbonyl (δ_{C} 181.7) was placed adjacent to the one at δ_{C} 181.5 based on evidence from the mass and UV spectra, which suggested the compound is an o-naphthoquinone. The presence of an aromatic benzene ring, a pyran ring, four sp² carbons and two carbonyls accounted for nine degrees of unsaturation. Hence, with no additional carbon signal, it was concluded that the carbonyl at δ_{C} 181.7 was directly linked to the aromatic ring thus accounting for the remaining one degree of unsaturation. Based on these spectral studies, a structure according to compound (2) was established for this new compound. Compound (2) is the first example in nature of an o-naphtho[1,8-bc]pyran quinoid with a 19 carbon skeleton.

20-Acetoxy-8-hydroxyserrulat-14-en-19-oic acid (compound (3)) is a pale yellow oil; [α] _{D}²⁵-63°(MeOH; c 0.268); λₘₐₓ^{MeOH} nm (log ε): 249 (3.9), 302 (3.4); μₘₐₓ (CH₂Cl₂) cm⁻¹: 3366, 2961, 2930, 1717, 1691, 1609, 1578; ¹H and ¹³C NMR data are shown in Table 3; HREIMS m/z: 374.2091 [M]⁺, LREIMS m/z: 374 [M]⁺, 314, 243, 230, 204, 159, 131, 109, 69. The molecular formula was determined to be C₂₂H₃₀O₅ from the molecular ion peak at m/z 374.2091 (Calcd at 374.2093) in the high resolution EI mass spectrum (HREIMS). This implied that the compound had eight degrees of unsaturation. The IR spectrum showed the presence of hydroxyl (3366 cm⁻¹), and carbonyl (1717 and 1691 cm⁻¹) functional groups. The ¹³C and the APT NMR spectra indicated a total of 22 carbons, four of which were methyls (δ_{C} 18.2, 19.6, 21.3 and 26.3), 5 methylenes with one directly attached to an oxygen atom (δ_{C} 21.0, 23.1, 27.7, 35.0 and 66.9), 3 sp² methines (δ_{C} 114.2, 123.6, and 126.1), 3 sp³ methines (δ_{C} 34.0, 39.6, and 44.2), 5 quaternary sp² carbons(δ_{C} 130.4, 130.8, 132.8, 144.2, and 156.9) and two carbonyls (171.0 and 173.7). In total, the 8 sp² carbons (four double bond functionalities) and the two carbonyls accounted for six degrees of unsaturation. This suggested that the compound contained two rings. The ¹H NMR spectrum showed signals for one acetyl methyl (δ_{H} 2.03, s), two methyl singlets (δ_{H} 1.52 and 1.62), a methyl doublet (δ_{H} 0.98, J=6.6 Hz), an oxy-methylene group (δ_{H} 4.32, dd, J=4.0 and 10.2 Hz; δ_{H} 4.06, br t, J=10.2 Hz), two aromatic protons (δ_{H} 7.39, br s and 7.25, d, J=1.8 Hz) suggesting the presence of an aromatic ring, a tri-substituted vinyl group with the olefinic proton resonating at 4.95 ppm (t br, J=7.0 Hz), and two benzylic protons (δ_{H} 2.66, m; 3.42, m). The benzylic proton shift at 3.42 ppm suggested the presence of a peri-hydroxyl group (Ghisalberti E L, 1995). The presence of the hydroxyl group was further supported by the appearance of the band at 3366 cm⁻¹ in the IR spectrum. In the COSY spectrum, the benzylic proton at δ_{H} 3.42 (δ_{C} 34.0) which showed vicinal coupling with the oxy-methylene protons δ_{H} 4.06 (¹H, t br, J=10.2 Hz; δ_{C} 66.9) and δ_{H} 4.32 (¹H, dd, J=10.2, 4.0 Hz; δ_{C} 66.9) also coupled to the methylene protons at δ_{H} 1.84, (m; δ_{C} 23.1). The protons of another methylene group (δ_{H} 1.83, m; 1.73, m; δ_{C} 21.0) exhibited coupling with the methylene protons at δ_{H} 1.84 and the benzylic proton at δ_{H} 2.66 (δ_{C} 44.2) thereby forming a six-membered carbocyclic ring. This indicated a bicyclic structure for the compound with the carbocyclic ring fused to an aromatic ring accounting for the remaining two degrees of unsaturation. The corresponding carbons bearing the different protons were identified from the HMQC spectrum. HMBC correlations between the two aromatic protons (δ_{H} 7.39, br s and 7.25, d, J=1.8 Hz) with the other sp² aromatic carbons and the carbonyl carbon at δ_{C} 171.0 (FIG. 2) indicated that they were located on either side of a quaternary carbon (δ_{C} 130.8) with a carbonyl substituent (δ_{C} 171.0). Also in the HMBC spectrum, the benzylic methine proton at δ_{H} 2.66 showed cross peaks with the methylene carbons at δ_{C} 23.1, δ_{H} 21.0, and δ_{H} 35.0; the quaternary carbons at δ_{C} 130.4 and δ_{C} 144.2; the aromatic sp² methine at δ_{C} 123.6 and the sp^{a} methine group at δ_{C} 39.6, while the other benzylic protons at δ_{H} 3.42 showed cross peaks with the oxy-methylene carbon at δ_{C} 66.9, the methylene carbon at δ_{H} 21.0 and the quaternary sp² carbons at δ_{C} 144.2 and 156.9, thus locating the aromatic ring. HMBC correlations of the aromatic moiety are shown in FIG. 2. The ¹³C NMR shift for the sp² carbon at δ_{C} 156.9 which also showed HMBC correlations with the aromatic protons at δ_{H} 7.25 was consistent with that of a phenolic carbon thus placing the phenolic hydroxyl group peri- to the benzylic proton at δ_{H} 3.42. HMBC interaction between the oxy-methylene protons and the acetyl carbonyl carbon at δ_{C} 173.7 which also correlated with the acetyl methyl protons at (δ_{H} 2.03 indicated that an acetoxy group was linked directly to the methylene carbon at δ_{C} 66.9. As there were no other signals in the ¹H and ¹³C NMR spectra for an alkoxy group, the carbonyl carbon, which showed HMBC cross peaks with the aromatic protons was considered to be the carbonyl of a carboxylic acid group. This is supported by the presence in the IR spectrum of the band at 1691 cm⁻¹. The other signals in the ¹H and ¹³C NMR spectra that also included a signal for an alkyl methine (δ_{H} 1.94, m, δ_{H} 39.6) and two methylenes (δ_{H} 1.26, m, 1.09, m, (δ_{H} 35.0 and δ_{H} 1.98, m, 1.84, m, δ_{H} 27.7) were assigned as a side chain connected to the bicyclic moiety. The COSY spectrum showed couplings of the olefinic proton (δ_{H} 4.95, t br, J=7.0 Hz; δ_{C} 126.1) to the methylene protons at δ_{H} 1.98, 1.84 (δ_{C} 27.7). These methylene protons coupled with the protons of the other methylene group at δ_{H} 1.26 and 1.09 ppm (δ_{C} 35.0), which in turn showed coupling with the alkyl methine proton. Interactions between the alkyl methine and the methyl protons at δ_{H} 0.98 (δ_{C} 19.6) were also observed in the COSY spectrum. The above couplings were also confirmed in the HMBC spectrum. HMBC correlations were also observed between the olefinic protons and the methyl carbons at 26.3 and 18.2 ppm. HMBC interactions of the alkyl methine proton with the methylene carbon at 21.0 ppm (δ_{H} 1.83, m, 1.73, m) and the quaternary aromatic carbon at δ_{C} 144.2 together with the coupling observed in the COSY spectrum between this alkyl methine proton and the benzylic proton at (δ_{H} 2.66 (δ_{C} 44.2) indicated that the alkyl methine group (and hence the side chain) was attached to the benzylic carbon. From the above spectral data, a structure according to compound (3) (FIG. 1) was established for this new compound. The relative stereochemistry of this compound was assumed to be the same as that of compound (4), a serrulatane diterpenoid whose relative stereochemistry had been shown to be the same as that of other serrulatane diterpenoids isolated from different Eremophila species (Forster P G et al., 1986). The structure of compound (4) is shown in FIG. 1. This assumption about the stereochemistry was made on the basis of the structural similarity existing between the two compounds. The serrulatanes represent the most common class of diterpenoids encountered so far in the genus Eremophila.

Bioactive Compounds from E. neglecta
a. Plant Material from E. neglecta

Leaves of E. neglecta were collected in northern South Australia, 119 km north of Marla.

### b. Isolation and Identification of Bioactive Compounds

The Et₂O extract of E. neglecta exhibited antimicrobial activity against S. aureus ATCC 29213 with a minimum inhibitory concentration (MIC) of 62.5 μg/mL. The n-hexane-, CH₂Cl₂-, and MeOH-soluble fractions obtained by partitioning the crude Et₂O extract were also examined for antimicrobial activity. Both the n-hexane- and the CH₂Cl₂-soluble fractions were found to be the most active, each having an MIC of 62.5 μg/mL.

### c. 2,19-diacetoxy-8-hydroxyserrulat-14-ene, 8,19-dihydroxyserrulat-14-ene and 8-hydroxyserrulat-14-en-19-oic acid

Bioassay-guided fractionation of a portion of the CH₂Cl₂ fraction led to the isolation of three compounds, 2,19-diacetoxy-8-hydroxyserrulat-14-ene (compound (6)), 8,19-dihydroxyserrulat-14-ene (compound (7)), and 8-hydroxyserrulat-14-en-19-oic acid (compound (8)) together with biflorin.

Compounds (7), (8) and biflorin isolated from E. neglecta showed antimicrobial activity against S. aureus (ATCC 29213) with MIC and MBC values ranging from 25.3 to 49.3 μM and 25.8 to 202.9 μM, respectively (data not shown). Compound (6) was the least active showing little activity at the maximum concentration tested (621.5 μM). Compounds (7), (8) and biflorin further showed antimicrobial activity against other Gram-positive bacteria including S. aureus (ATCC 25925), S. pyogenes, and S. pneumoniae. The MIC and MBC values are not shown for E. neglecta derived compounds. Again, little activity was observed for these compounds at the maximum concentrations tested against all Gram-negative bacteria tested.

The structures of compounds (6)-(8) were elucidated using 1D and 2D NMR spectroscopy, FT-IR, and mass spectrometry. Biflorin was identified by comparing its NMR and MS data with reported values (Fonseca A M et al., 2003).

8,19-dihydroxyserrulat-14-ene (subsequently found to correspond to the structure identified as compound (7)) was isolated as a pale yellow, amorphous solid. The molecular formula was determined to be C₂₀H₃₀O₂ from the molecular ion peak at m/z 302.2248 (calcd 302.2246) in the high-resolution EI mass spectrum (HREIMS). This implied that the compound had six degrees of unsaturation. The IR spectrum displayed absorption bands for hydroxy (3593 cm⁻¹) and CdC (1618 cm⁻¹) functional groups. The ¹³C and the APT NMR spectra (Table 4) indicated a total of 20 carbons, four of which were methyls (δ_{C} 17.6, 18.7, 21.0, and 25.7), five methylenes with one directly attached to an oxygen atom (δ_{C} 19.4, 26.2, 27.3, 33.5, and 65.4), three sp³ methines (δ_{C} 26.8, 38.0, and 42.5), three sp² methines (δ_{C} 111.1, 120.3, and 124.9), and five quaternary sp² carbons (δ_{C} 129.1, 131.2, 137.9, 141.5, and 153.6). All these resonances accounted for a partial molecular formula of C₂₀H₃₀O₂. The remaining two hydrogen atoms and one oxygen suggested the presence of hydroxy groups, as evidenced by the IR absorption band at 3593 cm⁻¹. The eight sp² carbons (four double-bond functionalities) accounted for four degrees of unsaturation. This suggested that the compound contained two rings. The ¹H NMR spectrum (Table 4) showed resonances for two methyl singlets (δ_{H} 1.63, br s; 1.53, br s), two methyl doublets (δ_{H} 1.18, d, J) 6.6 Hz; 0.94, d,) 6.6 Hz), an oxymethylene group (δ_{H} 4.56, br s), a trisubstituted vinyl group with the olefinic proton resonating at 4.96 ppm (t, J) 7.0 Hz), two aromatic protons that appeared as broad singlets but resolved into broad doublets upon Lorentzian/Gaussian resolution enhancement (Ferrige A G and Lindon J C, 1978) (δ_{H} 6.70, br d, J) 1.4 Hz; 6.64, br d, J) 1.4 Hz), and two benzylic protons (δ_{H} 3.09, d quint, J) 6.6, 3.0 Hz; 2.58, dt, J) 5.6, 3.0 Hz). The benzylic proton shift at 3.09 ppm suggested the presence of a peri-hydroxy group (Ghisalberti E L, 1995). The presence of the aromatic and benzylic protons indicated that the compound contained a benzene ring. In the COSY spectrum, the benzylic proton at δ_{H} 3.09 (δ_{C} 26.8), which showed vicinal coupling with a methyl doublet δhd H 1.18 (δ_{C} 21.0), also coupled to the methylene protons at (δ_{H} 1.88, m; 1.48, ddt, J) 13.0, 5.0, 3.0 Hz (δ_{C} 27.3). These methylene protons were connected to the benzylic proton at δ_{H} 2.58 (δ_{C} 42.5) by the methylene protons at (δ_{H} 1.86, m; 1.69, ddt, J) 13.5, 5.0, 3.0 Hz (δ_{C} 19.4), thereby forming a six-membered carbocyclic ring. This supported a bicyclic structure for the compound with the carbocyclic ring fused to an aromatic ring, thus accounting for the remaining two degrees of unsaturation. HMBC correlations between the two aromatic protons (δ_{H} 6.70, br d; 6.64, br d, each J) 1.4 Hz) with the other sp² aromatic carbons and the oxymethylene carbon (FIG. 1) indicated that they were located on either side of a quaternary carbon (δ_{C} 137.9) with an oxymethylene substituent (δ_{H} 4.56, br s, δ_{C} 65.4). In addition, the aromatic proton at (δ_{H} 6.64 showed a correlation with a quaternary aromatic carbon at δ_{C} 153.6 in the HMBC spectrum. The chemical shift of this carbon was consistent with that of a phenolic carbon. A HMBC correlation was also observed between this carbon and the benzylic proton at δ_{H} 3.09, placing the phenolic hydroxy group peri- to the benzylic proton.

TABLE 4
NMR Spectroscopic Data (600 MHz) for Compounds 2-4

| | 2^{a} | | 3^{b} | | 4^{b} | |
|---|---|---|---|---|---|---|
| Position | δ_{C}, mult. | δ_{H} (J in Hz) | δ_{C}, mult. | δ_{H} (J in Hz) | δ_{C}, mult. | δ_{H} (J in Hz) |
| 1 | 36.2, CH | 3.15 dq (6.6, 5.0) | 26.8, CH | 3.09 d quint (6.6, 3.0) | 27.3, CH | 3.18 d quint (7.0, 2.7) |
| 2 | 78.8, CH | 4.89 dt (8.7, 5.0) | 27.3, CH₂ | a 1.88 m; b 1.48 ddt | 27.0, CH₂ | a 1.95 m; b 1.52 m |
| | | | | (13.0, 5.0, 3.0) | | |
| 3 | 2.86, CH₂ | a 2.13 dt (13.0, 5.0); | 19.4, CH₂ | a 1.86 m; b 1.69 ddt | 19.2, CH₂ | a 1.89 m; b 1.73 m |
| | | b 1.45 dt (13.0, 8.7) | | (13.5, 5.0, 3.0) | | |
| 4 | 42.9, CH | 2.72 dt (8.7, 5.0) | 42.5, CH | 2.58 dt (5.6, 3.0) | 42.5, CH | 2.65 dt (5.7, 2.8) |
| 5 | 119.0, CH | 6.76 br s | 120.3, CH | 6.70 br d (1.4) | 124.1, CH | 7.54 br d (1.4) |
| 6 | 135.8, qC | | 137.9, qC | | 126.4, qC | |
| 7 | 113.9, CH | 6.63 br d (1.6) | 111.1, CH | 6.64 br d (1.4) | 113.2, CH | 7.29 br d (1.4) |
| 8 | 157.2, qC | | 153.6, qC | | 153.2, qC | |
| 9 | 129.5, qC | | 129.1, qC | | 136.5, qC | |
| 10 | 141.9, qC | | 141.5, qC | | 141.7, qC | |
| 11 | 35.3, CH | 2.27 m | 38.0, CH | 1.86 m | 38.8, CH | 1.89 m |
| 12 | 33.1, CH₂ | a 1.19 m; b 0.91 dddd | 33.5 CH₂ | a 1.27 dddd | 33.4, CH₂ | a 1.25 dddd |
| | | (13.5, 10.6, 8.5, 5.1) | | (13.0, 10.0, 7.0, 3.0); | | (13.0, 9.9, 7.1, 3.0); |
| | | | | b 1.09 dddd | | |
| | | | | (13.0, 10.0, 9.4, 5.0) | | |
| | | | | | | b 1.09 dddd |
| | | | | | | (13.0, 10.0, 9.4, 5.0) |
| 13 | 27.8, CH₂ | a 2.03 m; b 1.82 m | 26.2, CH₂ | a 1.97 m; b 1.79 m | 26.2, CH₂ | a 1.96 m; b 1.79 m |
| 14 | 126.3, CH | 5.07 t (6.6) | 124.9, CH | 4.96 t (7.0) | 124.6, CH | 4.96, t (7.0) |
| 15 | 132.9, qC | | 131.2, qC | | 131.4, qC | |
| 16 | 26.4, CH₃ | 1.67 br s | 25.7, CH₃ | 1.63 br s | 25.6, CH₃ | 1.62 br s |
| 17 | 18.2, CH₃ | 1.57 br s | 17.6, CH₃ | 1.53 br s | 17.6, CH₃ | 1.524 br s |
| 18 | 19.1, CH₃ | 1.05 d (6.6) | 18.7, CH₃ | 0.94 d (6.6) | 18.7, CH₃ | 0.96 d (6.6) |
| 19 | 68.1, CH₂ | 4.97 br s | 65.4, CH₂ | 4.56 br s | 172.1, qC | — |
| 20 | 20.2, CH₃ | 1.22 d (6.6) | 21.0, CH₃ | 1.18, d (6.6) | 20.8, CH₃ | 1.20 d (7.0) |
| 21 | 173.4, qC | | | | | |
| 22 | 21.8, CH₃ | 2.00 s | | | | |
| 23 | 173.1, qC | | | | | |
| 24 | 21.4, CH₃ | 2.05, s | | | | |

^{a}CD₃OD was used as solvent.
^{b}CDCl₃ was used as solvent.

The remaining resonances in the ¹H and ¹³C NMR spectra, which included a resonance for an alkyl methine (δ_{H} 1.86, m, δ_{C} 38.0) and two methylenes (δ_{H} 1.27, dddd, J) 13.0, 10.0, 7.0, 3.0 Hz; 1.09, dddd, J) 13.0, 10.0, 9.4, 5.0 Hz; δ_{C} 33.5 and δ_{H} 1.97, 1.79, m; δ_{C} 26.2), were assigned as a side chain connected to the bicyclic moiety. The COSY spectrum showed couplings of the alkyl methine proton to the methyl protons at δ_{H} 0.94 and the methylene protons at 1.27 and 1.09 ppm. The methylene protons at δ_{H} 1.97 and 1.79 (δ_{C} 26.2) connected the olefinic proton (δ_{H} 4.96, t, J) 7.0 Hz) to the methylene protons at 1.27 and 1.09 ppm. The above couplings were also confirmed in the HMBC spectrum. HMBC correlations were also observed between the olefinic protons and the methyl carbons at 25.7 and 17.6 ppm. HMBC correlations of the alkyl methine proton with the methylene carbon at 19.4 ppm (δ_{H} 1.86, m, 1.69, ddt J) 13.5, 5.0, 3.0 Hz) and the quaternary sp² carbon at δ_{C} 141.5 suggested that the methine group (and hence the side chain) was directly attached to the benzylic carbon at δ_{C} 42.5 ppm (δ_{H} 2.58, dt, J) 5.6, 3.0 Hz). This was confirmed by the presence of a significant ion at m/z 191 in the mass spectrum arising from the loss of the side chain (C₈H₁₅) from the molecular ion (m/z 302). This cleavage, referred to as the C-4/C-11 cleavage (Forster P G et al., 1986) is very common in the mass spectra of serrulatane diterpenoids. From the above data the structure of this new compound was established to be according to compound 7 (FIG. 1), and it was named 8,19-dihydroxyserrulat-14-ene.

It has been noted previously that it is difficult to assign the relative configuration in the serrulatane series on the basis of NMR data alone (Tippett L M and Massy-Westropp R A, 1993). The structure, absolute configuration, and the numbering system of the serrulatane skeleton 1 have been defined using X-ray crystallographic analysis. To date, all the different serrulatane diterpenoids that have been isolated from Eremophila species were shown to have the same configurational characteristics around C-1, C-4, and C-11. Consequently the relative configuration of these carbons for compound (7) was assumed to be the same as in structure for compound (1)(FIG. 1).

2,19-diacetoxy-8-hydroxyserrulat-14-ene (compound (6)) was isolated as a pale yellow oil. The molecular formula was determined to be C₂₄H₃₄O₅ from the molecular ion peak at m/z 402.2402 (calcd 402.2406) in the high-resolution EI mass spectrum (HREIMS). This indicated the presence of four additional carbons and three additional oxygens when compared to compound (7) and two additional degrees of unsaturation. The IR spectrum showed the presence of hydroxy (3582 cm⁻¹) and carbonyl (1732 cm⁻¹) functional groups. Analysis of the NMR spectroscopic data of compound (6) revealed that this compound was a serrulatane diterpenoid that was similar to compound (7). Its side chain was attached to the same position on the bicyclic ring as in (7), and the oxymethylene carbon at δ_{C} 68.1 (δ_{H} 4.97, br s) showed HMBC correlations with two aromatic protons at δ_{H} 6.76, br s, and 6.63, br d, J) 1.6 Hz. Similarly to compound (7), the aromatic proton at δ_{H} 6.63 showed an HMBC correlation to the phenolic quaternary carbon at δ_{C} 157.2. An HMBC correlation between this carbon and the benzylic proton at δ_{H} 3.15 placed the phenolic hydroxy group peri to this benzylic proton. The benzylic proton at δ_{H} 3.15 appeared as a broadened quintet in the ¹H NMR spectrum, but it resolved into a doublet of quartets upon Lorentzian/Gaussian resolution enhancement (Ferrige A G and Lindon J C, 1978) (dq, J) 6.6, 5.0 Hz). The substitution pattern on the aromatic ring for this compound was similar to that of compound (7) (FIG. 1). The major differences were the presence of resonances in the carbon NMR spectrum for two additional carbonyl carbons (δ_{C} 173.4 and 173.1) accounting for the additional two degrees of unsaturation. Extra resonances for two O-acetyl methyl singlets at δ_{H} 2.00, s, δ_{C} 21.8 and δ_{H} 2.05, s, δ_{C} 21.4 were also observed in the ¹H and ¹³C NMR spectra. The APT NMR spectrum for this compound indicated that it contained four sp³ methylene carbons and four sp³ methine carbons, ie one methylene carbon less and one methine carbon more when compared to compound (7). The presence of an oxymethine carbon at δ_{C} 78.8 (δ_{H} 4.89, dt, J) 8.7, 5.0 Hz) in addition to the oxymethylene carbon at δ_{C} 68.1 (δ_{H} 4.97, br s) in this compound suggested that a proton of one of the methylene groups in compound (7) had been substituted by an oxygen-containing group. An HMBC correlation between the oxymethine proton at δ_{H} 4.89 and the carbonyl carbon at δ_{C} 173.4 as well as between this carbonyl carbon and the O-acetyl methyl protons at δ_{H} 2.00 confirmed that the oxygen-containing group was in fact an acetoxy group. The coupling in the COSY spectrum between the oxymethine proton at δ_{H} 4.89, dt, J) 8.7, 5.0 Hz (δ_{C} 78.8) with the benzylic proton at δ_{H} 3.15 dq, J) 6.6, 5.0 Hz (δ_{C} 36.2) and also with the methylene protons at δ_{H} 2.13, dt, J) 13.0, 5.0 Hz and 1.45, dt, J) 13.0, 8.7 Hz (δ_{C} 28.6) located the acetoxy group at position 2 (Table 4). The appearance of the benzylic proton at δ_{H} 3.15 as a doublet of quartets in the ¹H NMR spectrum further confirms the attachment of the acetoxy group to C-2. The oxymethylene protons at δ_{H} 4.97, which showed HMBC correlations with the aromatic methine carbons at δ_{C} 119.0 and 113.9 (carbons connected to the aromatic protons), also showed an HMBC correlation with the carbonyl carbon at δ_{C} 173.1. In addition this carbonyl carbon showed an HMBC correlation with the O-acetyl methyl protons at δ_{H} 2.05. This indicated that compound (6) possessed a second acetoxy group, which was attached to the methylene group at C-19. Hence, structure given for compound (6) in FIG. 1 was established for this new compound. The configurational assignments at C-1, C-4, and C-11 were assumed to be the same as that of previous serrulatane diterpenoids isolated from other plant species in the genus Eremophila. The relative configuration at C-2 was determined by 2D ROESY NMR experiments. In the ROESY spectrum strong correlations were observed between H-2 and H-20, H-2 and H-4, H-20 and H-4, and also H-4 and H-18. This indicates that H-2 is cofacial with H-20, H-4, and H-18.

8-hydroxyserrulat-14-en-19-oic acid (compound (8)) was isolated as a white powder. The molecular formula was determined to be C₂₀H₂₈O₃ from the molecular ion peak at m/z 316.2035 (calcd 316.2038) in the HREIMS. This implied that the compound had seven degrees of unsaturation. The IR spectrum displayed absorption bands for hydroxy (3582 cm⁻¹), carbonyl (1726 cm⁻¹), and CdC (1688 cm⁻¹) functional groups. The ¹H and ¹³C NMR chemical shift assignments for this compound were very similar to those of the corresponding resonances for compound (7) and revealed the same structural features present in compound (7). The only major difference was the absence of the resonance of the oxymethylene group at δ_{H} 65.4 (δ_{H} 4.56, br s) present in compound (7). This resonance was replaced by a resonance for one carbonyl carbon (δ_{C} 172.1). The carbonyl carbon was considered to be the carbonyl of a carboxylic acid group, as there as an ion peak at m/z 271 in the mass spectrum, indicating the loss of a carboxylic acid group from the molecular ion (m/z 316). The presence of the carboxylic acid group accounted for the extra degree of unsaturation and the one additional oxygen atom present in this compound when compared to compound (7). HMBC correlations between the m-substituted aromatic protons (δ_{C} 7.54, br d, J) 1.4 Hz; δ_{H} 7.29, br d, J) 1.4 Hz) and the carbonyl carbon of the carboxylic acid group (FIG. 1) revealed that the carboxylic acid group was attached at the same position as the oxymethylene group in compound (7). This new compound was named 8-hydroxyserrulat-14-en-19-oic acid, and its configuration was assumed to be the same as that shown for compound (7).

### Example 2

### Methods and Materials

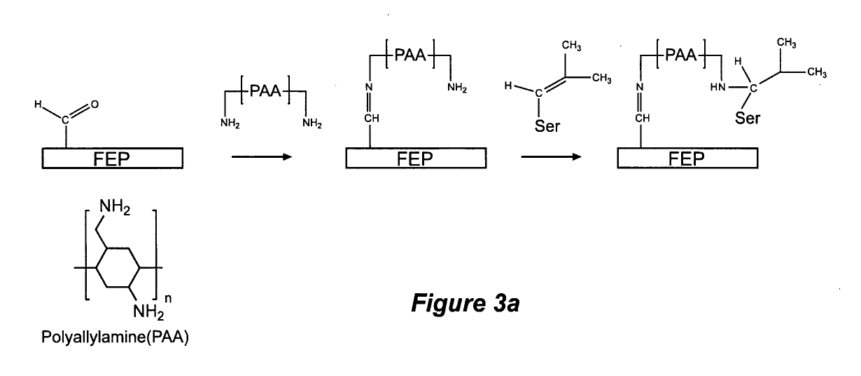

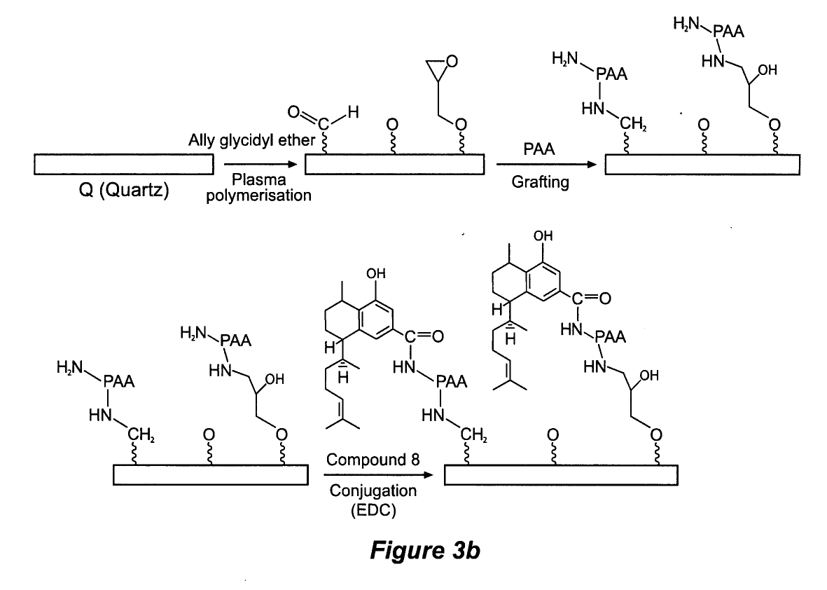

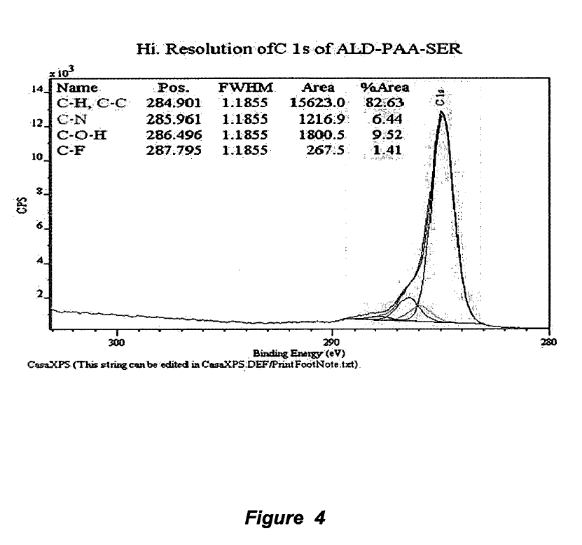

Preparation of Polymer Materials for Coating with Serrulatane Perfluorinated poly(ethylene-co-propylene)polymer (Teflon FEP, DuPont, 100A) provided a suitable test substrate for the coating of solid substrates in general as fluoropolymers are generally the most difficult class of polymers to be coated. Therefore, the coating strategies, represented schematically at FIG. 3 and described herein in detail, can be readily transferred to the preparation of many other substrate materials.

Initially, FEP samples were washed carefully to remove loose surface contamination. Analysis by X-ray photoelectron spectroscopy (XPS) (Kratos AXIS Ultra, monochromatic excitation) demonstrated an absence of contaminants, which might otherwise interfere with coating procedures and bacterial assays.

For substrate materials or devices that possess suitable reactive surface chemical groups such as amines, hydroxyls, or carboxyls, the following plasma coating step for activation of the surface should not be necessary. However, for surfaces without suitable reactive groups, an intermediate layer coating with functional (reactive) groups can be applied by plasma polymerisation, the deposition of a reactive plasma polymer layer, or by any other method well known to persons skilled in the art.

Preparation of Ceramic Materials for Coating with Serrulatanes

Silicon wafer pieces were used as a model ceramic substrate material. Serrulatanes cannot be immobilised directly onto ceramic substrate surfaces, therefore an intermediate layer to adhere to the ceramic surface and provide reactive surface groups for immobilising bioactive molecules thereto was formed. Several approaches for forming intermediate layers that adhere to ceramic and other surfaces and, which also provide reactive surface groups for immobilising bioactive molecules, are well known to persons skilled in the art. One approach is plasma polymerisation. Thus, silicon wafer pieces were washed carefully to remove loose surface contamination, as for FEP.

a. Plasma Polymerisation

Onto the chemically non-reactive FEP sheets or silicon wafer pieces, a thin polymeric interfacial bonding layer was applied by plasma polymerisation to provide the surface functional groups required for further covalent immobilisation reactions. Plasma polymerisation of propionaldehyde or allyl glycidyl ether was used for depositing the first layer, using a plasma reactor chamber powered by a 13.56 MHz generator. XPS analysis showed that this procedure produced a coating that possessed surface aldehyde groups, and surface epoxy and aldehyde groups, respectively, uniformly covering the surface of the FEP sheet or the silicon wafer piece. Subsequently, polyallylamine (PAA; MW 1500) was covalently grafted onto the surface aldehyde groups using a reductive amination reaction (see FIG. 3(a) and (b)). The sample was then immersed in an aqueous PAA solution (12 mg/ml) for 30 mins at room temperature, followed by sodium cyanoborohydride (NaCNBH₃) (12 mg/ml in aqueous solution) to convert the interfacial imine bonds to secondary amine linkages. The reaction mixture was kept at room temperature overnight. When using the allyl glycidyl ether plasma polymerised intermediate layer, cyanoborohydride reduction was not necessary. The slides were then rinsed three times with MilliQ water. The presence of a covalently attached layer of polyallylamine was verified by XPS analysis.

b. Plasma Deposition

Further FEP samples were coated using an alternative method. That is, surface amine groups were placed on FEP surfaces by the deposition of a thin (˜50 nm) plasma polymer layer fabricated from n-heptylamine vapour. This approach uses only a single step to prepare surfaces for the covalent attachment of serrulatane layers. The resultant surface is also less hydrophilic, which may have some bearing on certain biomedical applications.

### Covalent Binding of Active Serrulatane Compound

The subsequent method for the covalent immobilisation of serrulatane compounds is independent of the route by which amine surfaces were created.

a. Oxymercuration

Polyallylamine coated samples (prepared by either of the above methods) were placed in a 10 ml round bottom flask. 3 ml of tetrahydrofuran containing 57 mg of serrulatane compound and 27 mg of mercury (II) nitrate were added to the flask and sealed under a nitrogen atmosphere to avoid oxidative side reactions. The flask was agitated for 24 hours at 60° C. Afterwards, the solution inside the flask was removed and replaced with 3 ml of NaBH₄ solution (3 mg/ml in 10% NaOH). Next, the flask was agitated for 18 hours at 25° C. Then, the samples were washed with Milli-Q water and dried (see FIG. 3(a)).

b. Carbodiimide Catalysis

In an alternative covalent binding method, a serrulatane compound and 1-ethyl-3-(3-dimethylaminopropyl)carbodiimide hydrochloride (EDC, Sigma) were dissolved separately at 5 mg each in 1 ml of a 1:1 mixture of H₂O and ethanol or pure ethanol. Next, the pH of both solutions was adjusted to 4.4. Polyallylamine-coated FEP samples were placed in a vial, 3 ml of the serrulatane solution was added and then 3 ml of the EDC solution was added. The vial was kept in the refrigerator at 4° C. for 18 hrs. Subsequently, the samples were washed with Milli-Q water.

### Antimicrobial Assay of Serrulatane-Immobilised Surfaces

Bacterial assays on serrulatane-coated polymer samples were performed using Staphylococcus epidermis strain ATCC 35984. Samples were placed into wells of 12-well plates (disposable cell culture, Nunclon™ Surface, Denmark) and 2 ml of bacterial broth culture was introduced into each well. Broth culture comprised 10⁷ CFU/ml in Trypton Soy Broth (TSB)+0.25% glucose. The plates were incubated for one hour or four hours at 37° C. Then, the broth culture solution was removed from the wells and the wells washed twice with 2 ml of phosphate buffered saline. The samples in the wells were then immersed in 2 ml of 10% formaldehyde solution for 10 minutes to fix the surface-attached bacteria on the sample. After the fixing reagent had been removed, 2 ml of staining reagent (Bact Light LIVE/DEAD) was introduced into the wells and the plates were agitated gently for 15 minutes. Then using forceps, the stained samples were washed by immersion into saline solution and put onto glass slides for microscopic observation under a fluorescence microscope (Olympus BX 40). The fluorescence micrographs were analysed using AnalySis Five Olympus Imaging Software.

### Results

### Immobilisation of Serrulatane Compounds

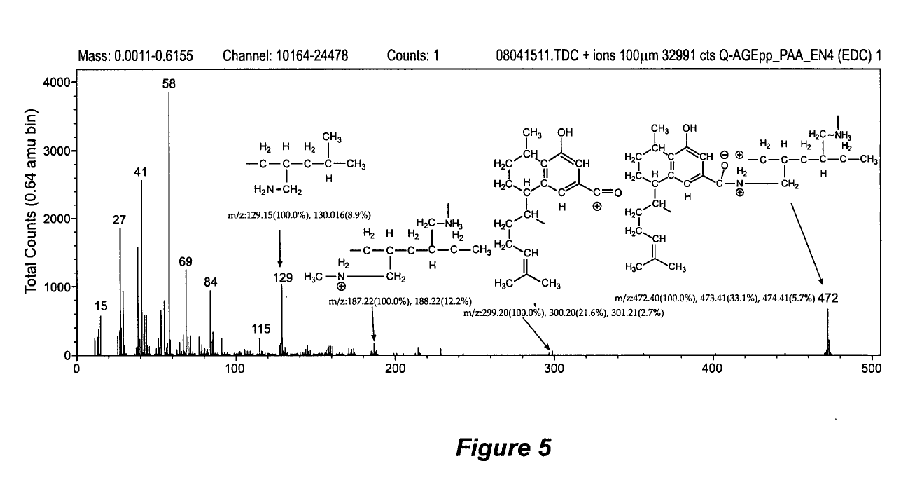

Samples with a covalently immobilised layer of serrulatane compounds were analysed by X-ray photoelectron spectroscopy (XPS) and Time-of-Flight Secondary Ion Mass Spectrometry (ToF-SIMS) and compared with reference spectra recorded on polyallylamine or heptylamine coated samples (without serrulatanes). The XPS and ToF-SIMS spectra clearly showed changes consistent with the attachment of a layer of diterpene compounds. FIG. 5 shows a ToF-SIMS spectrum recorded on a sample after immobilisation of a carboxylated serrulatane via amide bond formation. Characteristic mass signals assignable to serrulatane-derived ions indicate the presence of the compound on the surface, and polyallylamine-derived ions show the presence of the intermediate layer. The ion at m/z 472, in particular, shows the serrulatane molecule coupled via an amide linkage to a polyallylamine fragment, which indicates the success of the intended amide formation reaction. The covalent nature of the attachment was further investigated by washing with solvents intended to remove serrulatane molecules not covalently immobilised; such washing did not remove the peaks indicated, verifying the covalent nature of the immobilisation, which is essential for extended effectiveness and avoidance of diffusion away from the coated surface.

### Antimicrobial Activity of Surfaces

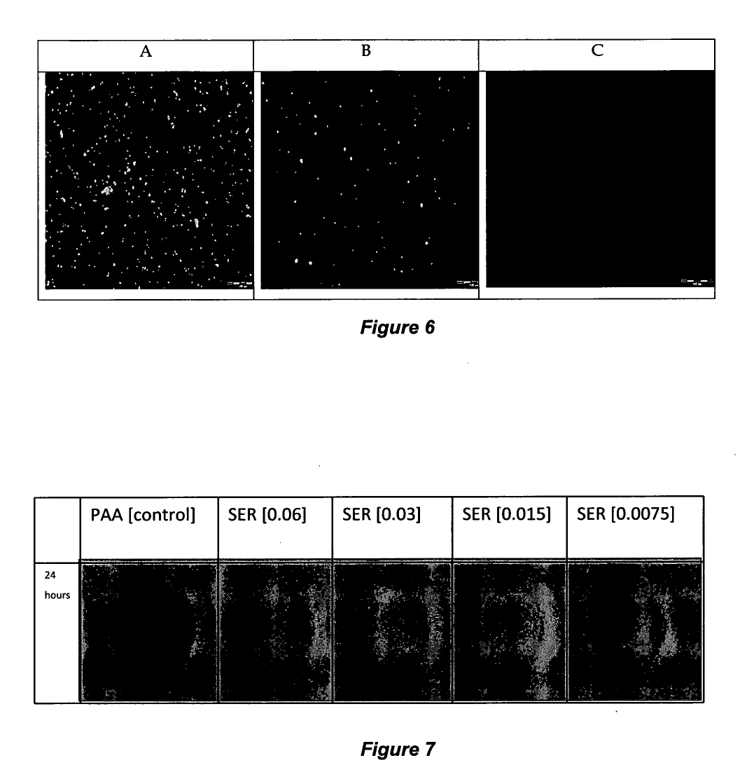

FIG. 6 shows fluorescent photomicrographs of bacterial colonisation of serrulatane+polyallylamine immobilised surfaces prepared as described above. Panel A shows a control surface, comprising a polyallylamine coating grafted onto a propanal plasma polymer intermediate layer. The extent of bacterial colonisation is indicated by green fluorescent staining (indicates viable bacteria), which shows substantial colonisation despite the presence of a protonated amine surface, which tends to be less hospitable to bacteria than many other polymeric surfaces.

Panel B and panel C show polyallylamine+serrulatane coated surfaces. The serrulatane compound was attached via oxymercuration (panel B) or via carbodiimide catalysis (panel C). After attachment of the serrulatane compound, the surfaces showed much reduced bacterial colonisation. The surface prepared by carbodiimide mediated attachment showed a complete elimination of bacterial colonisation, whereas the sample produced via oxymercuration showed some presence of viable bacteria. These results clearly indicate that surfaces prepared by either immobilisation method have antimicrobial activity. The difference in antimicrobial activity between surfaces is likely to be indicative of the need for further optimisation of the oxymercuration method (eg for reaction yield and surface coverage of attached serrulatane) rather than the greater effectiveness of the carbodiimide method.

Non-viable bacteria were indicated by red staining; very few non-viable bacteria were observed on any of the surfaces even under higher magnification.

Table 5 shows triplicate results obtained from surfaces coated by the carbodiimide method, and analysed by statistical analysis of multiple fields of view.

Resistance to biofilm formation was tested on each surface using extended bacterial culture conditions (24 and 48 hours) followed by staining of bacterial biofilms formed thereon with a 0.1% solution of safranin dye. FIG. 7 shows that the control polyallylamine surface became coated with a confluent biofilm within 24 hours, whereas the serrulatane coated surfaces showed bacteriostatic and/or bacteriocidal activity and remained free from bacterial growth and biofilm formation even when the surface coverage was diluted eight-fold.

Compatibility of Antimicrobial Surfaces with Mammalian Cells

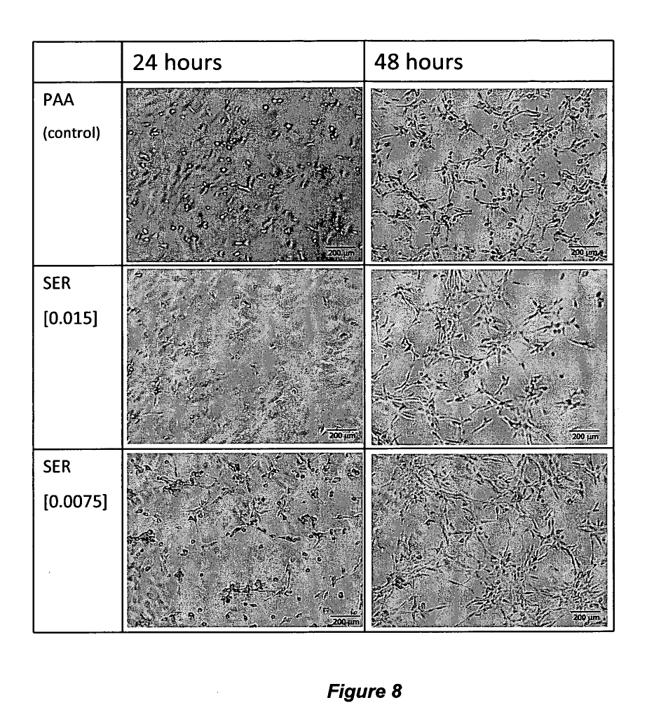

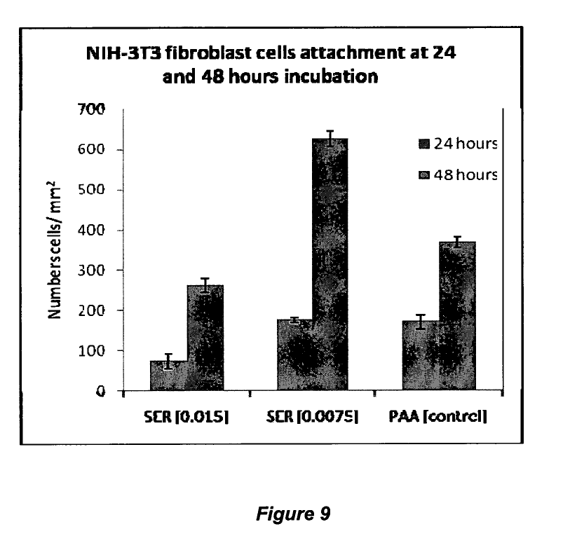

Minimal irritation of human cells and tissue is desirable in many implant situations. Compatibility with mammalian cells was tested using a mouse 3T3 fibroblast cell culture model, using standard cell culture techniques and photomicrographic assays of cell morphology and colonisation density well known to persons skilled in the art. FIG. 8 shows photomicrographs of fibroblast cells that had colonised the sample surfaces, after 24 and 48 hours. A low seeding density was employed in order to avoid confluent coverage that would obscure cell shape observation. On all three samples shown, the fibroblast cells were able to adhere and spread, and commenced to proliferate, albeit at different rates. FIG. 9 shows results collected by image analysis of a number of fields for each sample, and statistically evaluated. On the serrulatane coated samples, fibroblast cell numbers were equivalent or higher than on the polyallylamine control, indicating absence of deleterious effects of the serrulatane molecules on mammalian cells, whereas duplicate samples with the same coatings deterred bacterial colonisation as shown above. Therefore, it is possible to construct antimicrobial surfaces from serrulatane molecules such that adverse clinical reactions to the antibacterial coating on an implant should be tolerable.

TABLE 5
Bacterial attachment data on polymer samples coated with
serrulatane by the carbodiimide method

Viable

| Assays | Mean (n = 3 × 3) | Standard Deviation |
|---|---|---|
| PAA | 10.029 | 0.837 |
| Sample 1 | 0.175 | 0.100 |
| Sample 2 | 0.086 | 0.016 |
| Sample 3 | 0.633 | 0.221 |

A comparison of the viable bacterial counts of serrulatane-coated surface with the non-coated surface (PAA) indicates that the coated surface is resistant to bacterial colonisation. This result is consistent with the observations made following fluorescence staining and confirms that resistance to bacterial colonisation arises from the attachment of the antimicrobial compounds.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

All publications mentioned in this specification are herein incorporated by reference. Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed in Australia or elsewhere before the priority date of each claim of this application.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

### REFERENCES

1. Barr, A, Chapman, J, Smith, N, Wightman, G, Knight, T, Mills, L, Andrews, M, Alexander, V, 1993. Traditional Aboriginal medicines in the Northern Territory of Australia. Aboriginal communities of the Northern Territory. Conservation Commission of the Northern Territory, Darwin.
2. Chinnock, R J, 2007, Eremophila and Allied Genera. A Monograph of the Myoporaceae, Rosenberg Publishing, Kenthurst, New South Wales.
3. Darrouiche, R O, 2004, Treatment of infections associated with surgical implants, New England Journal of Medicine, 350, 1422.
4. Dehmel, F, Lex, J, and Schmalz, H G 2002 An approach to serrulatane diterpenes via endo-selective conjugate nucleophilic addition to arene-Cr(CO)(3) complexes, Organic Letters 4, 22, 3915-3918.
5. Ferrige, A G, Lindon, J C 1978 J. Magn. Reson. 31, 337-340.
6. Forster, P G, Ghisalberti, E L, Jefferies, P R, Poletti, V M, Whiteside, N J, 1986. Serrulatane diterpenoids from Eremophila spp. Phytochemistry 25, 1377-1383.
7. Fonseca, A M, Pessoa, O D L, Silveira, E R, Monte, F J Q, Braz-Filho, R, Lemos, T L G 2003 Magn. Reson. Chem. 41, 1038-1040.
8. Ghisalberti, E L, 1994a. The phytochemistry of the Myoporaceae. Phytochemistry 35, 7-33.
9. Ghisalberti, E L, 1995. The chemistry of unusual terpenoids from the genus Eremophila. Stud. Nat. Prod. Chem. 15, 225-287.
10. Ghisalberti, E L, 1994b. The ethnopharmacolgy and phytochemistry of Eremophila species (Myoporaceae). J. Ethnopharmacol. 44, 1-9.
11. Liu, Q, Harrington, D, Kohen, J L, Vemulpad, S, Jamie, J, 2006. Bactericidal and cyclooxygenase inhibitory diterpenes from Eremophila sturtii. Phytochemistry 67, 1256-1261.
12. Ndi, C P, Semple, S J, Griesser, H J, Barton, M D, 2007. Antimicrobial activity of some plant species from the Australian genus Eremophila. J. Basic Microbiol. 47, 158-164.
13. Ng, P S P and Banergee, A K, 2006. Alternative procedure for the synthesis of Mansonone F and Biflorin precursor, Natural Product Research, 20, 6, 629-635.
14. Oliver, R W A, Rashman, R M, 1971. Mass spectrometry of quinones. Part II. A study of the distinguishing features found in the mass spectra of 1,2- and 1,4-naphthoquinones. J. Chem. Soc. B, 341-344.
15. Palombo, E A, Semple, S J, 2001. Antibacterial activity of traditional Australian medicinal plants. J. Ethnopharmacol. 77, 151-157.
16. Shah, A, Cross, R F, Palombo, E A, 2004. Identification of the antibacterial component of an ethanolic extract of the Australian medicinal plant, Eremophila duttonii. Phytother. Res. 18, 615-618.
19. Smith, J E, Tucker, D, Watson, K, Jones, G L, 2007. J. Ethnopharmacol. 112, 386-393.
20. Tippett, L M, Massy-Westropp, R A, 1993. Phytochemistry 33, 417-421.
21. Thomson, R H, 1971. Naturally Occurring Quinones. Academic Press, New York.

1. A substrate comprising at least one surface wherein said surface comprises a compound according to Formula (I) immobilized thereto: 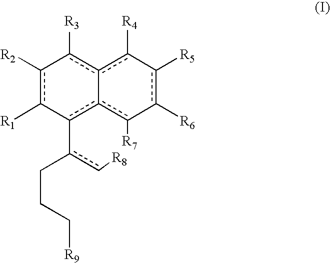
wherein
R₁, R₂, R₃, R₄, R₅, R₆ and R₉ are each independently selected from the group consisting of hydrogen, halogen, hydroxyl, formyl, alkyl, alkoxy, oxoalkyl, alkenyl, aryl, arylalkyl, carbonyl, carboxyl, aldehyde, carboxylate, cyano, ester, ether, carboxamide, amide, amine, sulfo, and sulfhydryl residues, their acids or salts, or derivatives thereof; and
R₇ and R₈ are each independently selected from the group consisting of hydrogen, halogen, hydroxyl, formyl, alkyl, alkoxy, oxoalkyl, alkenyl, carbonyl, carboxyl, aldehyde, carboxylate, cyano, ester, ether, carboxamide, amide, amine, sulfo, and sulfhydryl residues, their acids or salts, or derivatives thereof, or together form a C₁₋₃ or heteroatom bridge to form a cyclic or heterocyclic group.

2. The substrate according to claim 1, wherein at least one of R₁, R₂, R₃, R₄, R₅, R₆ and/or R₉ represent a linking residue(s) to the surface of the substrate.

3. The substrate according to claim 1, wherein the compound is according to Formula (II) or (III): 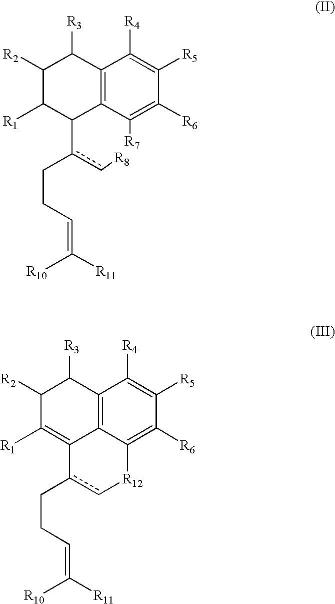
wherein
R₁, R₂, R₃, R₄, R₅, R₆, R₇, R₈, R₁₀ and R₁₁ are each independently selected from the group consisting of hydrogen, halogen, hydroxyl, formyl, alkyl, alkoxy, oxoalkyl, alkenyl, aryl, arylalkyl, carbonyl, carboxyl, aldehyde, carboxylate, cyano, ester, ether, carboxamide, amide, amine, sulfo, and sulfhydryl residues, their acids or salts, or derivatives thereof and
R₁₂ is selected from the group consisting of C₁₋₃ alkyl, C₁₋₃ alkoxy, C₁₋₃ oxoalkyl, C₁₋₃ alkenyl, C₁₋₃ carbonyl, C₁₋₃ carboxyl, C₁₋₃ aldehyde, C₁₋₃ carboxylate, C₁₋₃ cyano, C₁₋₃ ester, C₁₋₃ ether, C₁₋₃ carboxamide, C₁₋₃ amide, C₁₋₃ amine, NH, O, sulfo and sulfhydryl residues, or derivatives thereof.

4. The substrate according to claim 3, wherein at least one of R₁, R₂, R₃, R₄, R₅, R₆, R₁₀ and/or R₁₁ represent a linking residue(s) to the surface of the substrate.

5. The substrate according to claim 4, wherein residue R₁, R₂, R₃, R₄, R₅, R₆, R₁₀ and/or R₁₁ is covalently linked to the surface of the substrate.

6. The substrate according to claim 5 comprising less than about 10 mg of the compound per cm² of substrate surface.

7. A device comprising a substrate according to claim 1.

8. A device according to claim 7, wherein said device is a medical device.

9. A device according to claim 8, wherein said substrate comprises a thin film perfluorinated poly(ethylene-co-propylene)polymer (FEP).

10. A method for immobilizing a compound according to Formula (I): 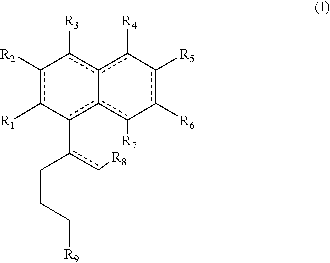
wherein
R₁, R₂, R₃, R₄, R₅, R₆ and R₉ are each independently selected from the group consisting of hydrogen, halogen, hydroxyl, formyl, alkyl, alkoxy, oxoalkyl, alkenyl, aryl, arylalkyl, carbonyl, carboxyl, aldehyde, carboxylate, cyano, ester, ether, carboxamide, amide, amine, sulfo, and sulfhydryl residues, their acids or salts, or derivatives thereof; and
R₇ and R₈ are each independently selected from the group consisting of hydrogen, halogen, hydroxyl, formyl, alkyl, alkoxy, oxoalkyl, alkenyl, aryl, alkyaryl, carbonyl, carboxyl, aldehyde, carboxylate, cyano, ester, ether, carboxamide, amide, amine, sulfo, and sulfhydryl residues, their acid or salts, or derivatives thereof, or together form a C₁₋₃ or heteroatom bridge to thereby form a cyclic or heterocyclic group;
to at least one surface of a substrate, said method comprising the steps of:
(i) optionally preparing the surface of the substrate to be reactive with the compound; and
(ii) reacting the compound with the surface so as to immobilize the compound onto the surface.

11. The method according to claim 10, wherein step (i) comprises coating the surface of the substrate with an intermediate layer comprising surface functional groups.

12. The method according to claim 11, wherein the intermediate layer is formed by deposition of a thin plasma polymer layer, and step (ii) comprises covalently immobilizing the compound to the surface of the substrate.

13. The method according to claim 10, wherein the compound is covalently immobilized onto the surface.

14. A method for immobilizing a compound according to Formula (I): 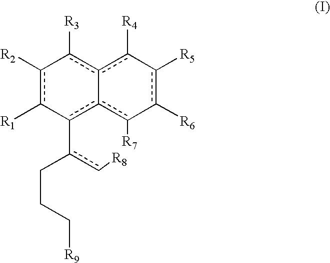
wherein
R₁, R₂, R₃, R₄, R₅, R₆ and R₉ are each independently selected from the group consisting of hydrogen, halogen, hydroxyl, formyl, alkyl, alkoxy, oxoalkyl, alkenyl, aryl, arylalkyl, carbonyl, carboxyl, aldehyde, carboxylate, cyano, ester, ether, carboxamide, amide, amine, sulfo, and sulfhydryl residues, their acids or salts, or derivatives thereof; and
R₇ and R₈ are each independently selected from the group consisting of hydrogen, halogen, hydroxyl, formyl, alkyl, alkoxy, oxoalkyl, alkenyl, aryl, alkyaryl, carbonyl, carboxyl, aldehyde, carboxylate, cyano, ester, ether, carboxamide, amide, amine, sulfo, and sulfhydryl residues, their acids or salts, or derivatives thereof, or together form a C₁₋₃ or heteroatom bridge to thereby form a cyclic or heterocyclic group;
to at least one surface of a substrate, said method comprising covalently immobilizing the compound to said surface by thin plasma polymer deposition.

15. A compound according to Formula (IV): 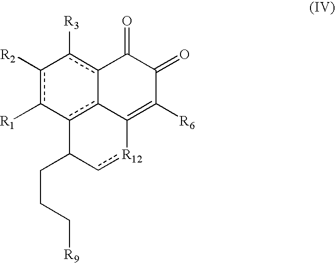
wherein
R₁, R₂, R₃ and R₆ are each independently selected from the group consisting of hydrogen, halogen, hydroxyl, formyl, alkyl, alkoxy, oxoalkyl, alkenyl, aryl, arylalkyl, carbonyl, carboxyl, aldehyde, carboxylate, cyano, ester, ether, carboxamide, amide, amine, sulfo, and sulfhydryl residues, their acids or salts or derivatives thereof,
R₉ and R₁₂ are each independently selected from the group consisting of C₁₋₃ hydroxyl, C₁₋₃ alkyl, C₁₋₃ alkoxy, C₁₋₃ oxoalkyl, C₁₋₃ alkenyl, C₁₋₃ carbonyl, C₁₋₃ carboxyl, C₁₋₃ aldehyde, C₁₋₃ carboxylate, C₁₋₃ cyano, C₁₋₃ ester, C₁₋₃ ether, C₁₋₃ carboxamide, C₁₋₃ amide, C₁₋₃ amine, NH, O, sulfo and sulfhydryl residues, or derivatives thereof, and
wherein said compound is other than naphtho(1,8-bc)pyran-7,8-dione.

16. The compound of claim 15, wherein said compound is also other than 9-methyl-3-(4-methyl-3-pentenyl)-2,3-dihydronaphthol[1,8-bc]-pyran-7,8-dione.

17. The compound of claim 15, wherein said compound is according to Formula (V): 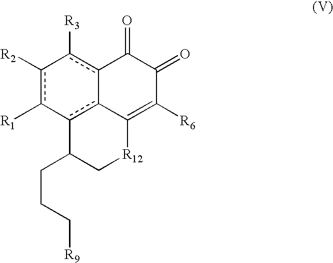
or Formula (VI): 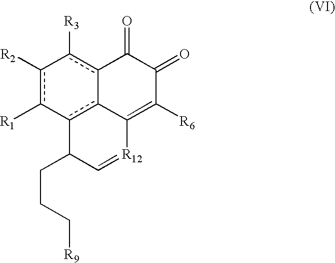
wherein R₁₂ is selected from the group consisting of C₁₋₃ hydroxyl, C₁₋₃ alkyl, C₁₋₃ alkoxy, C₁₋₃ oxoalkyl, C₁₋₃ alkenyl, C₁₋₃ carbonyl, C₁₋₃ carboxyl, C₁₋₃ aldehyde, C₁₋₃ carboxylate, C₁₋₃ cyano, C₁₋₃ ester, C₁₋₃ ether, C₁₋₃ carboxamide, C₁₋₃ amide, C₁₋₃ amine, NH, sulfo and sulfhydryl residues, or derivatives thereof.

18. The compound of claim 17, wherein R₉ is: 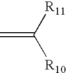
wherein
R₁₀ and R₁₁ are independently selected from the group consisting of hydrogen, halogen, hydroxyl, formyl, alkyl, alkoxy, oxoalkyl, alkenyl, aryl, arylalkyl, carbonyl, carboxyl, aldehyde, carboxylate, cyano, ester, ether, carboxamide, amide, amine, sulfo, and sulfhydryl residues, their acids or salts or derivatives thereof,

19. The compound of claim 15, wherein said compound is according to Formula (VII): 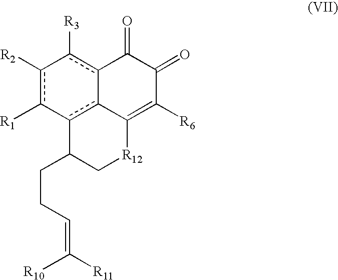
wherein
R₁, R₂, R₃, R₆, R₁₀ and R₁₁ are each independently selected from the group consisting of hydrogen, halogen, hydroxyl, formyl, alkyl, alkoxy, oxoalkyl, alkenyl, aryl, arylalkyl, carbonyl, carboxyl, aldehyde, carboxylate, cyano, ester, ether, carboxamide, amide, amine, sulfo, and sulfhydryl residues, their acids or salts or derivatives thereof and
wherein R₁₂ is selected from the group consisting of C₁₋₃ alkyl, C₁₋₃ alkoxy, C₁₋₃ oxoalkyl, C₁₋₃ alkenyl, C₁₋₃ carbonyl, C₁₋₃ carboxyl, C₁₋₃ aldehyde, C₁₋₃ carboxylate, C₁₋₃ cyano, C₁₋₃ ester, C₁₋₃ ether, C₁₋₃ carboxamide, C₁₋₃ amide, C₁₋₃ amine, NH, O, sulfo and sulfhydryl residues, or derivatives thereof.

20. The compound of claim 15, wherein said compound is selected from the group consisting of: 9-methyl-3-(4-methyl-3-pentenyl)-2,3-dihydronaphthol[1,8-bc]-pyran-7,8-dione, 20-acetoxy-8-hydroxyserrulat-14-en-19-oic acid, 8,19-dihydroxyserrulat-14-ene and 8-hydroxyserrulat-14-en-19-oic acid.
